(19) United States
(12) Patent Application Publication
(10) Pub. No.: US 2008/0306107 A1
(43) Pub. Date: Dec. 11, 2008

(54) Compounds

(75) Inventors: Andrew Griffin, Montreal (CA); Andrea Penwell, Montreal (CA); Miroslaw Tomaszewski, Montreal (CA); Simon Woo, Montreal (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/721,637

(22) PCT Filed: Dec. 22, 2005
(86) PCT No.: PCT/SE2005/002020

§ 371 Date: Mar. 14, 2008

(30) Foreign Application Priority Data
Dec. 23, 2004 (SE) 0403171-2

(51) Int. Cl. A61K 31/4365; C07D 495/04; C07D 213/57; A61P 29/00

(52) U.S. Cl. 514/301; 546/114; 546/286

(57) ABSTRACT

The present invention relates to new compounds of formula (I) wherein R¹ to R⁹, X, p and n are defined as in claim 1, or salts, solvates or solvated salts thereof, processes for their preparation and to new intermediates used in the preparation thereof, pharmaceutical compositions containing said compounds and to the use of said compounds in therapy.

### FIELD OF THE INVENTION

The present invention relates to new compounds, to pharmaceutical compositions containing said compounds and to the use of said compounds in therapy. The present invention further relates to processes for the preparation of said compounds and to new intermediates used in the preparation thereof.

### BACKGROUND OF THE INVENTION

Pain sensation in mammals is due to the activation of the peripheral terminals of a specialized population of sensory neurons known as nociceptors. Capsaicin, the active ingredient in hot peppers, produces sustained activation of nociceptors and also produces a dose-dependent pain sensation in humans. Cloning of the vanilloid receptor 1 (VR1 or TRPV1) demonstrated that VR1 is the molecular target for capsaicin and its analogues. (Caterina, M. J., et al., et. al. Nature (1997) v. 389 p 816-824). Functional studies using VR1 indicate that it is also activated by noxious heat, tissue acidification) and other inflammatory mediators (Tominaga, M., et. al. Neuron (1998) v. 21, p. 531-543). Expression of VR1 is also regulated after peripheral nerve damage of the type that leads to neuropathic pain. These properties of VR1 make it a highly relevant target for pain and for diseases involving inflammation. While agonists of the VR1 receptor can act as analgesics through nociceptor destruction, the use of agonists, such as capsaicin and its analogues, is limited due to their pungency, neurotoxicity and induction of hypothermia. Instead, agents that block the activity of VR1 should prove more useful. Antagonists would maintain the analgesic properties, but avoid pungency and neurotoxicity side effects.

Compounds with VR1 inhibitor activity are believed to be of potential use for the treatment and/or prophylaxis of disorders such as pain, especially that of inflammatory or traumatic origin such as arthritis, ischaemia, fibromyalgia, low back pain and post-operative pain (Walker et al., J Pharmacol Exp Ther. (2003) January; 304(1):56-62). In addition to this visceral pains such as chronic pelvic pain, cystitis, irritable bowel syndrome (IBS), pancreatitis and the like, as well as neuropathic pain such as sciatia, diabetic neuropathy, HIV neuropathy, multiple sclerosis, and the like (Walker et al ibid, J Pharmacol Exp Ther. (2003) March; 304(3):940-8), are potential pain states that could be treated with VR1 inhibition. These compounds are also believed to be potentially useful for inflammatory disorders like asthma, cough, inflammatory bowel disease (IBD) (Hwang, et al., Curr Opin Pharmacol (2002) June; 2(3):235-42). Compounds with VR1 blocker activity are also useful for itch and skin diseases like psoriasis and for gastro-esophageal reflux disease (GERD), emesis, urinary incontinence and hyperactive bladder (Yiangou et al BJU Int (2001) June; 87(9):774-9, Szallasi, Am J Clin Pathol (2002) 118: 110-21). VR1 inhibitors are also of potential use for the treatment and/or prophylaxis of the effects of exposure to VR1 activators like capsaicin or tear gas, acids or heat (Szallasi ibid).

The role for VR1 antagonists in Inflammatory Bowel Diseases (IBD) is further supported by the finding that primary sensory neuron denervation by subcutaneous administration of capsaicin to neonatal rats, resulted in decreased levels of disease activity index (DAI), MPO and histological damage to the gut in DSS colitis model compared to control (N Kihara, et al., Gut, 2003. 52: p: 713-719). TRPV1 antagonists attenuate macroscopic symptoms in DSS colitis model in mice (E. S. KIMBALL, et al., Neurogastroenterol Motil, 2004. 16: p. 1-8).

The potential for a role for VR1 antagonists in Irritable Bowel Syndrome (IBS) has been described. Patients with faecal urgency and rectal hypersensitivity have increased levels of TRPV1 expression in nerve fibres in muscle, submucosal and mucosal layers. This also correlates with increase sensitivity to heat and distension (C L H Chan, et al., THE LANCET, 2003. 361 (February 1): p. 385-91). Jejunal wide dynamic range (WDR) afferents show lower firing in response to pressure ex vivo in TRPV1−/− mice (Rong W, H. K., et al., J Physiol (Lond). 2004. 560: p. 867-881). The visceromotor responses to jejunal and colorectal distension in rat are affected by a TRPV1 antagonist using both ramp and phasic distensions (Winchester, EMG response to jejunal and colorectal distension in rat are affected by a TRPV1 antagonist in both ramp and phasic distensions. DDW abstract, 2004). Capsaicin applied to the ileum induce pain and mechanical hyperalgesia in human experimental model (Asbjφorn Mohr Drewes, et al., Pain, 2003. 104: p. 333-341).

A role in Gastroesophageal Reflux Disease (GERD) for VR1 antagonists has been mentioned in the literature. Patients with oesophagitis have increased levels of TRPV1 expression in peripheral nerves enervating the oesophageal epithelium (P. J. Matthews, et al., European J. of Gastroenterology & Hepatology, 2004. 16: p. 897-902). Even if the TRPV1 antagonist JYL1421 only has minor effects of acid-induced excitation of esophageal afferents, an antagonist with a different profile has yet to be evaluated. Since TRPV1 appears to play a role in mechanosensation, it is possible that antagonists may inhibit TLESRs, the main cause of gastroesophageal reflux.

A further potential use relates to the treatment of tolerance to VR1 activators.

VR1 inhibitors may also be useful in the treatment of interstitial cystitis and pain related to interstitial cystitis.

### PRIOR ART

Guerrera, et al., describe the synthesis and antifungal activity of pyrido[3′,2′:4,5]thieno[3,2-d]-1,2,3-triazine derivatives. (Farmaco (1993), 48(12), 1725-33).

Dunn, A., et al., disclose a nucleophilic displacements in pyridine rings. (J. of Heterocyclic Chemistry (1987), 24(1), 85-9)

Tornetta, B., et al., disclose the synthesis and spectral behavior of pyridothienoisothiazole and pyridothienopyrimidine derivatives. (Gazzetta Chimica Italiana (1978), 108 (1-2), 57-62)

Guerrera, F.; et al., further discloses the synthesis of 3-aminothieno[2,3-b]pyridine derivatives, pyridothienopyrimidine and pyridothienoisothiazole derivatives. (Chimica e l'Industria (Milan, Italy), (1976), 58(6), 451-2.)

Schneller, S., et al., describe fused thieno[3,2-d]-v-triazine-4 (3H)-ones in Heterocycles (1975), 3(2), 135-8.

### DETAILED DESCRIPTION OF THE INVENTION

The object of the present invention is to provide compounds) exhibiting an inhibitory activity at the vanilloid receptor 1 (VR1).

The present invention provides a compound of formula I

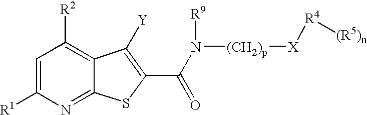

wherein:
R¹ and R² are independently selected from H, NO₂, NH₂, halo, N(C₁₋₃alkyl)₂, C₁₋₃alkyl, C₂₋₃alkenyl, C₂₋₃alkynyl, C₁₋₃haloalkyl, C₁₋₃haloalkylO, hydroxyC₁₋₃alkyl, C₁₋₃alkylOC₀₋₃alkyl, C₁₋₃alkylSC₀₋₃alkyl and C₁₋₃alkylNC₀₋₃alkyl;

### Y is NH₂, NH(R³), N(R³)₂, OH, OR³ or NO₂;

R³ is C₁₋₃alkyl, C₂₋₃alkenyl, C₂₋₃alkynyl, C₁₋₃haloalkyl, C₁₋₃haloalkylO, hydroxyC₁₋₃alkyl, C₁₋₃alkylOC₀₋₃alkyl, C₁₋₃alkylSC₀₋₃alkyl or C₁₋₃alkylNC₀₋₃alkyl;
R⁹ is H, C₁₋₆alkyl, R⁶OC₀₋₆alkyl, or C₅₋₁₀arylC₀₋₆alkyl;
X is bond, CR⁶R⁷, NR⁶R⁷ or O;
p is 0, 1, 2, or 3;
R⁴ is bond, H, C₁₋₆alkyl, C₂₋₆alkenyl, C₂₋₆alkynyl, C₁₋₆haloalkyl, C₁₋₆haloalkylO, C₅₋₁₀arylC₀₋₆alkyl, C₅₋₁₀heteroarylC₀₋₆alkyl, C₃₋₁₅cycloalkylC₀₋₆alkyl, C₃₋₁₅heterocycloalkylC₀₋₆alkyl, R⁶OC₀₋₆alkyl, R⁶SC₀₋₆alkyl or R⁶NC₀₋₆alkyl, COOR⁶, R⁶COR⁷, R⁶CO₂, R⁶CONR⁷R⁸, R⁶NR⁷COC₀₋₆alkyl, R⁶SO₂R⁷ or R⁶SOR⁷R⁸;
R⁵ is H; OH, oxy, NO₂, NH₂, halo, N(C₁₋₃alkyl)₂, C₁₋₃alkyl, C₂₋₃alkenyl, C₂₋₃alkynyl, C₁₋₃haloalkyl, C₁₋₃haloalkylO, hydroxyC₁₋₃alkyl, R⁶OC₀₋₆alkyl, R⁶SC₀₋₆alkyl, R⁶NC₀₋₆alkyl, C₅₋₁₀-arylOC₀₋₆alkyl, C₅₋₁₀ heteroarylOC₀₋₆alkyl, C₃₋₁₀-cycloalkylOC₀₋₆alkyl, R⁶COO, R⁶COR⁷, R⁶CO₂, R⁶CONR⁷R⁸, R⁶NR⁷COC₀₋₆alkyl or R⁶SO₂R⁷ or R⁶SOR⁷R⁸;
R⁶, R⁷ and R⁸ are independently selected from H, C₁₋₆alkyl and C₅₋₁₀arylC₀₋₆alkyl;
or X and R⁶ form a 4, 5, 6 or 7 membered ring; and
n is 0, 1, 2, 3, 4, 5, 6 or 7;
or salts, solvates or solvated salts thereof.

In one embodiment of the invention R¹ is C₁₋₂alkyl. In another embodiment R¹ is methyl, ethyl, n-propyl or i-propyl.

In a further embodiment R² is C₁₋₂haloalkyl, whereby halo is fluoro or bromo. In one embodiment R² is fluoromethyl, difluoromethyl, trifluoromethyl, fluoroethyl or difluoroethyl. In yet another embodiment R² is trifluoromethyl.

In one embodiment Y is NH₂ or NH(R³), wherein R³ is C₁₋₃alkyl. In another embodiment Y is NH₂.

In yet another embodiment R⁹ is H or C₁₋₆alkyl. In yet a further embodiment R⁹ is H. In one embodiment R⁹ is methyl, ethyl, n-propyl or i-propyl.

In a further embodiment of the invention X is a bond. In another embodiment X is CR⁶R⁷, whereby R⁶ and R⁷ may the same or different and selected from H, C₁₋₃alkyl and C₅₋₁₀arylC₀₋₃alkyl. In one embodiment X is NR⁶R⁷ and O. In another embodiment X is methyl. In yet another embodiment R⁶ and X form together phenyl.

In one embodiment R⁴ is C₅₋₁₀arkylC₀₋₆alkyl or C₁₋₆alkyl. In a further embodiment R⁴ is C₅₋₆aryl.

In yet a further embodiment R⁴ is phenyl.

In one embodiment R⁵ is H, halo, C₁₋₃alkyl, C₁₋₃haloalkyl or R⁶OC₀₋₆alkyl.

In another embodiment R⁵ is H, chloro or fluoro.

In a further embodiment R⁵ is C₁₋₃alkyl. In yet another embodiment R⁵ is methyl, ethyl, n-propyl or i-propyl.

In yet a further embodiment R⁵ is C₁₋₂haloalkyl, whereby halo is fluoro or bromo.

In one embodiment R⁵ is fluoromethyl, difluoromethyl, trifluoromethyl, fluoroethyl or difluoroethyl. In yet another embodiment R⁵ is trifluoromethyl.

In another embodiment R⁵ is R⁶OC₀₋₆alkyl, whereby R⁶ is C₁₋₃alkyl. In a further embodiment R⁶ is methoxy, ethoxy or propoxy.

In one embodiment p is 1, 2, or 3, with the proviso that the compound is not 3-amino-6-methyl-4-trifluoromethyl-thieno[2,3-b]pyridine-2-carboxylic acid benzylamide.

Another embodiment of the invention relates to the compound selected from the group consisting of
3-amino-6-methyl-N-(3-phenylpropyl)-4-(trifluoromethyl)thieno[2,3-b]pyridine-2-carboxamide,
3-amino-6-methyl-N-[2-(4-methylphenyl)ethyl]-4-(trifluoromethyl)thieno[2,3-b]pyridine-2-carboxamide,
3-amino-6-methyl-N-[2-(2-methylphenyl)ethyl]-4-(trifluoromethyl)thieno[2,3-b]pyridine-2-carboxamide,
3-amino-6-methyl-N-(2-phenylpropyl)-4-(trifluoromethyl)thieno[2,3-b]pyridine-2-carboxamide,
3-amino-N,6-dimethyl-N-(2-phenylethyl)-4-(trifluoromethyl)thieno[2,3-b]pyridine-2-carboxamide,
3-amino-N-[2-(2-methoxyphenyl)ethyl]-6-methyl-4-(trifluoromethyl)thieno[2,3-b]pyridine-2-carboxamide,
3-amino-N-(2,2-diphenylethyl)-6-methyl-4-(trifluoromethyl)thieno[2,3-b]pyridine-2-carboxamide,
3-amino-N-[2-(3-fluorophenyl)ethyl]-6-methyl-4-(trifluoromethyl)thieno[2,3-b]pyridine-2-carboxamide,
3-amino-N-[2-(3,4-dichlorophenyl)ethyl]-6-methyl-4-(trifluoromethyl)thieno[2,3-b]pyridine-2-carboxamide,
3-amino-6-methyl-4-(trifluoromethyl)-N-{2-[3-(trifluoromethyl)phenyl]ethyl}thieno[2,3-b]pyridine-2-carboxamide,
3-amino-6-methyl-N-{2-[3-(methyloxy)phenyl]ethyl}-4-(trifluoromethyl)thieno[2,3-b]pyridine-2-carboxamide,
3-amino-6-methyl-N-[2-(2-thienyl)ethyl]-4-(trifluoromethyl)thieno[2,3-b]pyridine-2-carboxamide,
3-amino-N-[2-(2,6-dichlorophenyl)ethyl]-6-methyl-4-(trifluoromethyl)thieno[2,3-b]pyridine-2-carboxamide,
3-amino-N-[2-(2-fluorophenyl)ethyl]-6-methyl-4-(trifluoromethyl)thieno[2,3-b]pyridine-2-carboxamide,
3-amino-6-methyl-N-[2-(phenyloxy)ethyl]-4-(trifluoromethyl)thieno[2,3-b]pyridine-2-carboxamide,
3-amino-N-(2,3-dihydro-1,4-benzodioxin-2-ylmethyl)-6-methyl-4-(trifluoromethyl)thieno[2,3-b]pyridine-2-carboxamide,
3-amino-N-{2-[4-(ethyloxy)phenyl]ethyl}-6-methyl-4-(trifluoromethyl)thieno[2,3-b]pyridine-2-carboxamide,
3-amino-6-methyl-N-(4-methylcyclohexyl)-4-(trifluoromethyl)thieno[2,3-b]pyridine-2-carboxamide,
3-amino-6-methyl-N-{2-[2-(phenyloxy)phenyl]ethyl}-4-(trifluoromethyl)thieno[2,3-b]pyridine-2-carboxamide,
3-amino-6-methyl-N-{[5-methyl-2-(trifluoromethyl)-3-furanyl]methyl}-4-(trifluoromethyl)thieno[2,3-b]pyridine-2-carboxamide,
1,1-dimethylethyl 4-({[3-amino-6-methyl-4-(trifluoromethyl)thieno[2,3-b]pyridin-2-yl]carbonyl}amino)-1-piperidinecarboxylate,
3-amino-N-{[3-fluoro-5-(trifluoromethyl)phenyl]methyl}-6-methyl-4-(trifluoromethyl)thieno[2,3-b]pyridine-2-carboxamide,
3-amino-6-methyl-4-(trifluoromethyl)-N-{[3-(trifluoromethyl)phenyl]methyl}thieno[2,3-b]pyridine-2-carboxamide,
3-amino-N-(3,3-dimethylbutyl)-6-methyl-4-(trifluoromethyl)thieno[2,3-b]pyridine-2-carboxamide,
3-amino-6-methyl-4-(trifluoromethyl)-N-({3-[(trifluoromethyl)oxy]phenyl}methyl)thieno[2,3-b]pyridine-2-carboxamide,
3-amino-N-{2-[4-(1,1-dimethylethyl)phenyl]ethyl}-6-methyl-4-(trifluoromethyl)thieno[2,3-b]pyridine-2-carboxamide,
3-amino-6-methyl-N-{3-[methyl(phenyl)amino]propyl}-4-(trifluoromethyl)thieno[2,3-b]pyridine-2-carboxamide,
3-amino-N-[(3,5-dimethylphenyl)methyl]-6-methyl-4-(trifluoromethyl)thieno[2,3-b]pyridine-2-carboxamide,
3-amino-N-(cyclohexylmethyl)-6-methyl-4-(trifluoromethyl)thieno[2,3-b]pyridine-2-carboxamide,
3-amino-N-butyl-6-methyl-4-(trifluoromethyl)thieno[2,3-b]pyridine-2-carboxamide,
3-amino-N-[2-(2,4-dichlorophenyl)ethyl]-6-methyl-4-(trifluoromethyl)thieno[2,3-b]pyridine-2-carboxamide,
3-amino-N-cyclohexyl-6-methyl-4-(trifluoromethyl)thieno[2,3-b]pyridine-2-carboxamide,
3-amino-N-[(5-fluoro-2-methylphenyl)methyl]-6-methyl-4-(trifluoromethyl)thieno[2,3-b]pyridine-2-carboxamide,
3-amino-N-[1-(4-fluorophenyl)ethyl]-6-methyl-4-(trifluoromethyl)thieno[2,3-b]pyridine-2-carboxamide,
3-amino-6-methyl-N-(2-methylpropyl)-4-(trifluoromethyl)thieno[2,3-b]pyridine-2-carboxamide,
3-amino-N-[(6-fluoro-4H-1,3-benzodioxin-8-yl)methyl]-6-methyl-4-(trifluoromethyl)thieno[2,3-b]pyridine-2-carboxamide,
3-amino-N,6-dimethyl-4-(trifluoromethyl)-N-{[3-(trifluoromethyl)phenyl]methyl}thieno[2,3-b]pyridine-2-carboxamide,
3-amino-N-(2,3-dihydro-1-benzofuran-5-ylmethyl)-6-methyl-4-(trifluoromethyl)thieno[2,3-b]pyridine-2-carboxamide,
3-amino-6-methyl-N-[2-(2-pyridinyl)ethyl]-4-(trifluoromethyl)thieno[2,3-b]pyridine-2-carboxamide,
3-amino-6-methyl-N-[2-(4-pyridinyl)ethyl]-4-(trifluoromethyl)thieno[2,3-b]pyridine-2-carboxamide,
3-amino-6-methyl-N-[(2S)-2-phenylpropyl]-4-(trifluoromethyl)thieno[2,3-b]pyridine-2-carboxamide,
3-amino-6-methyl-N-[(2R)-2-phenylpropyl]-4-(trifluoromethyl)thieno[2,3-b]pyridine-2-carboxamide,
3-amino-N-[(2R)-2-hydroxy-2-phenylethyl]-6-methyl-4-(trifluoromethyl)thieno[2,3-b]pyridine-2-carboxamide,
3-amino-N-[(2S)-2-hydroxy-2-phenylethyl]-6-methyl-4-(trifluoromethyl)thieno[2,3-b]pyridine-2-carboxamide,
3-amino-N-(2-hydroxy-2-phenylpropyl)-6-methyl-4-(trifluoromethyl)thieno[2,3-b]pyridine-2-carboxamide,
3-amino-N-[2-(2-furyl)ethyl]-6-methyl-4-(trifluoromethyl)thieno[2,3-b]pyridine-2-carboxamide,
3-amino-N-[2-(4-fluorophenyl)ethyl]-6-methyl-4-(trifluoromethyl)thieno[2,3-b]pyridine-2-carboxamide,
3-amino-N-(2-cyclohexylethyl)-6-methyl-4-(trifluoromethyl)thieno[2,3-b]pyridine-2-carboxamide,
3-amino-6-methyl-N-(trans-4-methylcyclohexyl)-4-(trifluoromethyl)thieno[2,3-b]pyridine-2-carboxamide,
6-methyl-3-(methylamino)-N-(2-phenylethyl)-4-(trifluoromethyl)thieno[2,3-b]pyridine-2-carboxamide,
3-(dimethylamino)-6-methyl-N-(2-phenylethyl)-4-(trifluoromethyl)thieno[2,3-b]pyridine-2-carboxamide,
3-amino-N-(2-phenylethyl)-4-(trifluoromethyl)thieno[2,3-b]pyridine-2-carboxamide,
3-amino-N-[2-(4-methylphenyl)ethyl]-4-(trifluoromethyl)thieno[2,3-b]pyridine-2-carboxamide, and
3-amino-N-[2-(3-fluorophenyl)ethyl]-4-(trifluoromethyl)thieno[2,3-b]pyridine-2-carboxamide,
   or salts, solvates or solvated salts thereof.

A further embodiment of the invention relates to the compound selected from the group consisting of
3-amino-6-methyl-N-(3-phenylpropyl)-4-(trifluoromethyl)thieno[2,3-b]pyridine-2-carboxamide,
3-amino-6-methyl-N-[2-(4-methylphenyl)ethyl]-4-(trifluoromethyl)thieno[2,3-b]pyridine-2-carboxamide,
3-amino-6-methyl-N-[2-(2-methylphenyl)ethyl]-4-(trifluoromethyl)thieno[2,3-b]pyridine-2-carboxamide,
3-amino-6-methyl-N-(2-phenylpropyl)-4-(trifluoromethyl)thieno[2,3-b]pyridine-2-carboxamide,
3-amino-N,6-dimethyl-N-(2-phenylethyl)-4-(trifluoromethyl)thieno[2,3-b]pyridine-2-carboxamide,
3-amino-N-[2-(2-methoxyphenyl)ethyl]-6-methyl-4-(trifluoromethyl)thieno[2,3-b]pyridine-2-carboxamide,
3-amino-N-(2,2-diphenylethyl)-6-methyl-4-(trifluoromethyl)thieno[2,3-b]pyridine-2-carboxamide,
3-amino-N-[2-(3-fluorophenyl)ethyl]-6-methyl-4-(trifluoromethyl)thieno[2,3-b]pyridine-2-carboxamide,
3-amino-N-[2-(3,4-dichlorophenyl)ethyl]-6-methyl-4-(trifluoromethyl)thieno[2,3-b]pyridine-2-carboxamide,
3-amino-6-methyl-4-(trifluoromethyl)-N-{2-[3-(trifluoromethyl)phenyl]ethyl}thieno[2,3-b]pyridine-2-carboxamide, and
   or salts, solvates or solvated salts thereof.

A yet further embodiment of the invention relates to the compound selected from the group consisting of
3-amino-6-methyl-N-{2-[3-(methyloxy)phenyl]ethyl}-4-(trifluoromethyl)thieno[2,3-b]pyridine-2-carboxamide,
3-amino-6-methyl-N-[2-(2-thienyl)ethyl]-4-(trifluoromethyl)thieno[2,3-b]pyridine-2-carboxamide,
3-amino-N-[2-(2,6-dichlorophenyl)ethyl]-6-methyl-4-(trifluoromethyl)thieno[2,3-b]pyridine-2-carboxamide,
3-amino-N-[2-(2-fluorophenyl)ethyl]-6-methyl-4-(trifluoromethyl)thieno[2,3-b]pyridine-2-carboxamide,
3-amino-6-methyl-N-[2-(phenyloxy)ethyl]-4-(trifluoromethyl)thieno[2,3-b]pyridine-2-carboxamide,
3-amino-N-(2,3-dihydro-1,4-benzodioxin-2-ylmethyl)-6-methyl-4-(trifluoromethyl)thieno[2,3-b]pyridine-2-carboxamide,
3-amino-N-{2-[4-(ethyloxy)phenyl]ethyl}-6-methyl-4-(trifluoromethyl)thieno[2,3-b]pyridine-2-carboxamide,
3-amino-6-methyl-N-(4-methylcyclohexyl)-4-(trifluoromethyl)thieno[2,3-b]pyridine-2-carboxamide,
3-amino-6-methyl-N-{2-[2-(phenyloxy)phenyl]ethyl}-4-(trifluoromethyl)thieno[2,3-b]pyridine-2-carboxamide,
3-amino-6-methyl-N-{[5-methyl-2-(trifluoromethyl)-3-furanyl]methyl}-4-(trifluoromethyl)thieno[2,3-b]pyridine-2-carboxamide,
1,1-dimethylethyl 4-({[3-amino-6-methyl-4-(trifluoromethyl)thieno[2,3-b]pyridin-2-yl]carbonyl}amino)-1-piperidinecarboxylate,
3-amino-N-{[3-fluoro-5-(trifluoromethyl)phenyl]methyl}-6-methyl-4-(trifluoromethyl)thieno[2,3-b]pyridine-2-carboxamide,
3-amino-6-methyl-4-(trifluoromethyl)-N-{[3-(trifluoromethyl)phenyl]methyl}thieno[2,3-b]pyridine-2-carboxamide,
3-amino-N-(3,3-dimethylbutyl)-6-methyl-4-(trifluoromethyl)thieno[2,3-b]pyridine-2-carboxamide,
3-amino-6-methyl-4-(trifluoromethyl)-N-({3-[(trifluoromethyl)oxy]phenyl}methyl)thieno[2,3-b]pyridine-2-carboxamide,
3-amino-N-{2-[4-(1,1-dimethylethyl)phenyl]ethyl}-6-methyl-4-(trifluoromethyl)thieno[2,3-b]pyridine-2-carboxamide,
3-amino-6-methyl-N-{3-[methyl(phenyl)amino]propyl}-4-(trifluoromethyl)thieno[2,3-b]pyridine-2-carboxamide,
3-amino-N-[(3,5-dimethylphenyl)methyl]-6-methyl-4-(trifluoromethyl)thieno[2,3-b]pyridine-2-carboxamide,
3-amino-N-(cyclohexylmethyl)-6-methyl-4-(trifluoromethyl)thieno[2,3-b]pyridine-2-carboxamide,
3-amino-N-butyl-6-methyl-4-(trifluoromethyl)thieno[2,3-b]pyridine-2-carboxamide,
3-amino-N-[2-(2,4-dichlorophenyl)ethyl]-6-methyl-4-(trifluoromethyl)thieno[2,3-b]pyridine-2-carboxamide,
3-amino-N-cyclohexyl-6-methyl-4-(trifluoromethyl)thieno[2,3-b]pyridine-2-carboxamide,
3-amino-N-[(5-fluoro-2-methylphenyl)methyl]-6-methyl-4-(trifluoromethyl)thieno[2,3-b]pyridine-2-carboxamide,
3-amino-N-[1-(4-fluorophenyl)ethyl]-5-methyl-4-(trifluoromethyl)thieno[2,3-b]pyridine-2-carboxamide,
3-amino-6-methyl-N-(2-methylpropyl)-4-(trifluoromethyl)thieno[2,3-b]pyridine-2-carboxamide,
3-amino-N-[(6-fluoro-4H-1,3-benzodioxin-8-yl)methyl]-6-methyl-4-(trifluoromethyl)thieno[2,3-b]pyridine-2-carboxamide,
3-amino-N,6-dimethyl-4-(trifluoromethyl)-N-{[3-(trifluoromethyl)phenyl]methyl}thieno[2,3-b]pyridine-2-carboxamide,
3-amino-N-(2,3-dihydro-1-benzofuran-5-ylmethyl)-6-methyl-4-(trifluoromethyl)thieno[2,3-b]pyridine-2-carboxamide,
3-amino-6-methyl-N-[2-(2-pyridinyl)ethyl]-4-(trifluoromethyl)thieno[2,3-b]pyridine-2-carboxamide, and
3-amino-6-methyl-N-[2-(4-pyridinyl)ethyl]-4-(trifluoromethyl)thieno[2,3-b]pyridine-2-carboxamide,
   or salts, solvates or solvated salts thereof.

Yet another embodiment of the invention relates to the compounds selected from the group consisting of
3-amino-6-methyl-N-[(2S)-2-phenylpropyl]-4-(trifluoromethyl)thieno[2,3-b]pyridine-2-carboxamide,
3-amino-6-methyl-N-[(2R)-2-phenylpropyl]-4-(trifluoromethyl)thieno[2,3-b]pyridine-2-carboxamide,
3-amino-N-[(2R)-2-hydroxy-2-phenylethyl]-6-methyl-4-(trifluoromethyl)thieno[2,3-b]pyridine-2-carboxamide,
3-amino-N-[(2S)-2-hydroxy-2-phenylethyl]-6-methyl-4-(trifluoromethyl)thieno[2,3-b]pyridine-2-carboxamide,
3-amino-N-(2-hydroxy-2-phenylpropyl)-6-methyl-4-(trifluoromethyl)thieno[2,3-b]pyridine-2-carboxamide,
3-amino-N-[2-(2-furyl)ethyl]-6-methyl-4-(trifluoromethyl)thieno[2,3-b]pyridine-2-carboxamide,
3-amino-N-[2-(4-fluorophenyl)ethyl]-6-methyl-4-(trifluoromethyl)thieno[2,3-b]pyridine-2-carboxamide,
3-amino-N-(2-cyclohexylethyl)-6-methyl-4-(trifluoromethyl)thieno[2,3-b]pyridine-2-carboxamide,
3-amino-6-methyl-N-(trans-4-methylcyclohexyl)-4-(trifluoromethyl)thieno[2,3-b]pyridine-2-carboxamide,
6-methyl-3-(methylamino)-N-(2-phenylethyl)-4-(trifluoromethyl)thieno[2,3-b]pyridine-2-carboxamide,
3-(dimethylamino)-6-methyl-N-(2-phenylethyl)-4-(trifluoromethyl)thieno[2,3-b]pyridine-2-carboxamide,
3-amino-N-(2-phenylethyl)-4-(trifluoromethyl)thieno[2,3-b]pyridine-2-carboxamide,
3-amino-N-[2-(4-methylphenyl)ethyl]-4-(trifluoromethyl)thieno[2,3-b]pyridine-2-carboxamide, and
3-amino-N-[2-(3-fluorophenyl)ethyl]-4-(trifluoromethyl)thieno[2,3-b]pyridine-2-carboxamide,
   or salts, solvates or solvated salts thereof.

For the avoidance of doubt it is to be understood that in this specification ‘C₁₋₆’ means a carbon group having 1, 2, 3, 4, 5 or 6 carbon atoms.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, t-pentyl, neo-pentyl, n-hexyl or i-hexyl, t-hexyl. The term C₁₋₃ alkyl having 1 to 3 carbon atoms and may be methyl, ethyl, n-propyl, i-propyl or tert-butyl.

The term ‘C₀’ means a bond or does not excist. For example when R¹ is C₀alkyl, R¹ is a bond and “arylC₀alkyl” is equivalent with “aryl”, “C₂alkylOC₀alkyl” is equivalent with “C₂alkylO”.

In this specification, unless stated otherwise, the term “alkenyl” includes both straight and branched chain alkenyl groups. The term “C₂₋₆alkenyl” having 2 to 6 carbon atoms and one or two double bonds, may be, but is not limited to vinyl, allyl, propenyl, butenyl, crotyl, pentenyl, or hexenyl, and a butenyl group may for example be buten-2-yl, buten-3-yl or buten-4-yl.

In this specification, unless stated otherwise, the term “alkynyl” includes both straight and branched chain alkynyl groups. The term “C₂₋₆alkynyl” having 2 to 6 carbon atoms and one or two triple bonds, may be, but is not limited to etynyl, propargyl, pentynyl or hexynyl and a butynyl group may for example be butyn-3-yl or butyn-4-yl.

In this specification, unless stated otherwise, the term “cycloalkyl” refers to an optionally substituted, saturated cyclic hydrocarbon ring system. The term “C₃₋₇cycloalkyl” may be cyclopropyl, cyclobutyl, cyclopentyl, cyclohexyl or cycloheptyl.

In this specification, unless stated otherwise, the term “heterocycloalkyl” refers to a 3- to 7-membered, non-aromatic, partially or completely saturated hydrocarbon group, which contains one ring and at least one heteroatom. Examples of said heterocycle include, but are not limited to pyrrolidinyl, pyrrolidonyl, piperidinyl, piperazinyl, morpholinyl, oxazolyl, 2-oxazolidonyl or tetrahydrofuranyl.

In this specification, unless stated otherwise, the term “aryl” refers to an optionally substituted monocyclic or bicyclic hydrocarbon unsaturated aromatic ring system. Examples of “aryl” may be, but are not limited to phenyl and naphthyl.

In this specification, unless stated otherwise, the term “heteroaryl” refers to an optionally substituted monocyclic or bicyclic unsaturated aromatic ring system containing at least one heteroatom selected independently form N, O or S. Examples of “heteroaryl” may be, but are not limited to pyridyl, pyrrolyl, furyl, thienyl, imidazolyl, oxazolyl, isoxazolyl, thiazolyl, pyrazolyl, benzofuryl, indolyl, isoindolyl, benzimidazolyl, pyridazinyl, pyrimidinyl, pyrazinyl, tetrazolyl, triazolyl or oxazolyl.

In this specification, unless stated otherwise, the terms “arylalkyl” and “heteroarylalkyl” refer to a substituent that is attached via the alkyl group to an aryl or heteroaryl group.

In this specification, unless stated otherwise, the term “4, 5, 6 or 7 membered ring” includes aryl, heteroaryl, cycloalkyl and heterocycloalkyl as defined above.

In this specification, unless stated otherwise, the terms “halo” and “halogen” may be fluoro, iodo, chloro or bromo.

In this specification, unless stated otherwise, the term “haloalkyl” means an alkyl group as defined above, which is substituted with halo as defined above. The term “C₁₋₆haloalkyl” may include, but is not limited to fluoromethyl, difluoromethyl, trifluoromethyl, fluoroethyl, difluoroethyl or bromopropyl. The term “C₁₋₆haloalkylO” may include, but is not limited to fluoromethoxy, difluoromethoxy, trifluoromethoxy, fluoroethoxy or difluoroethoxy.

The present invention relates to the compounds of formula I as hereinbefore defined as well as to the salts, solvates or solvated salts thereof. Salts for use in pharmaceutical formulations will be pharmaceutically acceptable salts, but other salts may be useful in the production of the compounds of formula I.

A suitable pharmaceutically acceptable salt of the compounds of the invention is, for example, an acid-addition salt, for example a salt with an inorganic or organic acid. In addition, a suitable pharmaceutically acceptable salt of the compounds of the invention is an alkali metal salt, an alkaline earth metal salt or a salt with an organic base.

Other pharmaceutically acceptable salts and methods of preparing these salts may be found in, for example, Remington's Pharmaceutical Sciences (18^{th} Edition, Mack Publishing Co.).

Some compounds of formula I may have chiral centres and/or geometric isomeric centres (E- and Z-isomers), and it is to be understood that the invention encompasses all such optical, diastereoisomeric and geometric isomers.

The invention also relates to any and all tautomeric forms of the compounds of formula I.

### Medical Use

Surprisingly, it has been found that the compounds according to the present invention are useful in therapy. The compounds of formula I, or salts, solvates or solvated salts thereof, as well as their corresponding active metabolites, exhibit a high degree of potency and selectivity for individual vanilloid receptor 1 (VR1) groups. Accordingly, the compounds of the present invention are expected to be useful in the treatment of conditions associated with excitatory activation of vanilloid receptor 1 (VR1).

The compounds may be used to produce an inhibitory effect of VR1 in mammals, including man.

VR1 are highly expressed the peripheral nervous system and in other tissues. Thus, it is expected that the compounds of the invention are well suited for the treatment of VR1 mediated disorders.

The compounds of formula I are expected to be suitable for the treatment of acute and chronic pain, acute and chronic neuropathic pain and acute and chronic inflammatory pain. Examples of such disorder may be selected from the group comprising arthritis, rheumatoid arthritis, spondylitis and gout, fibromyalgia, low back pain and sciatica, post-operative pain, cancer pain, migraine and tension headache, visceral pains like chronic pelvic pain, cystitis, including interstitial cystitis, pancreatitis, renal and biliary colic, menstruation associated pain, pain related to ischeamic and angina, neuropathic pain disorders such as diabetic neuropathy, HIV neuropathy, chemotherapy induced neuropathies, post-herpetic neuralgia, post traumatic neuralgia and complex regional syndrome as well as itch.

Further relevant disorders may be selected from the group comprising gastro-esophageal reflux disease (GERD), functional gastrointestinal disorders (FGD) such as irritable bowel syndrome (IBS), irritable bowel syndrome (IBS), and functional dyspepsia (FD).

Further examples of disorders are overactive bladder (“OAB”), a term for a syndrome that encompasses urge incontinence, urgency and frequency. Compounds of the invention may alleviate urinary incontinence (“UI”) the involuntary loss of urine that results from an inability of the bladder to retain urine as a consequence of either urge (urge incontinence), or physical or mental stress (stress incontinence).

Other relevant disorders may be psoriasis, and emesis.

Yet further relevant disorders are related to respiratory diseases and may be selected from the group comprising cough, asthma, chronic obstructive lung disease and emphysema, lung fibrosis and interstitial lung disease.

The VR1 inhibitor(s) for respiratory use, may be administrated by either an oral or inhaled route. The respiratory disease may be an acute and chronic illness and may be related to infection(s) and/or exposure to environmental pollution and/or irritants.

The compounds of formula I may also be used as antitoxin to treat (over-) exposure to VR1 activators like capsaicin, tear gas, acids or heat. Regarding heat, there is a potential use for VR1 antagonists in (sun-)burn induced pain, or inflammatory pain resulting from burn injuries.

The compounds may further be used for treatment of tolerance to VR1 activators.

One embodiment of the invention relates to the use of the compounds of formula I as hereinbefore defined, in therapy.

Another embodiment of the invention relates to the use of the compounds of formula I as hereinbefore defined, for treatment of VR1 mediated disorders.

A further embodiment of the invention relates to the use of the compounds of formula I as hereinbefore defined, for treatment of acute and chronic pain.

Yet another embodiment of the invention relates to the use of the compounds of formula I as hereinbefore defined, for treatment of acute and chronic neuropathic pain.

Yet a further embodiment of the invention relates to the use of the compounds of formula I as hereinbefore defined, for treatment of acute and chronic inflammatory pain.

One embodiment of the invention relates to the use of the compounds of formula I as hereinbefore defined, for treatment of arthritis, rheumatoid arthritis, spondylitis and gout, fibromyalgia, low back pain and sciatica, post-operative pain, cancer pain, migraine and tension headache, visceral pains like chronic pelvic pain, cystitis, including interstitial cystitis, pancreatitis, renal and biliary colic, menstruation associated pain, pain related to ischeamic and angina, neuropathic pain disorders such as diabetic neuropathy, HIV neuropathy, chemotherapy induced neuropathies, post-herpetic neuralgia, post traumatic neuralgia and complex regional syndrome as well as itch.

Another embodiment of the invention relates to the use of the compounds of formula I as hereinbefore defined, for treatment of gastro-esophageal reflux disease, functional gastrointestinal disorders, irritable bowel syndrome, irritable bowel syndrome and functional dyspepsia.

A further embodiment of the invention relates to the use of the compounds of formula I as hereinbefore defined, for treatment of overactive bladder.

Yet a further embodiment of the invention relates to the use of the compound of formula I as hereinbefore defined, for the treatment of respiratory diseases selected from the group comprising of cough, asthma, chronic obstructive lung disease and emphysema, lung fibrosis and interstitial lung disease.

One embodiment of the invention relates to the use of the compound of formula I as hereinbefore defined, in the manufacture of a medicament for treatment of VR1 mediated disorders and for treatment of acute and chronic pain, acute and chronic neuropathic pain and acute and chronic inflammatory pain, and respiratory diseases, and any other disorder mentioned above.

Another embodiment of the invention relates to a method of treatment of VR1 mediated disorders and acute and chronic pain, acute and chronic neuropathic pain and acute and chronic inflammatory pain, and respiratory diseases, and any other disorder mentioned above, comprising administering to a mammal, including man in need of such treatment, a therapeutically effective amount of the compounds of formula I, as hereinbefore defined.

A further embodiment of the invention relates to a pharmaceutical composition comprising a compound of formula I as hereinbefore defined, for use in treatment of VR1 mediated disorders and for treatment of acute and chronic pain, acute and chronic neuropathic pain and acute and chronic inflammatory pain, and respiratory diseases, and any other disorder mentioned above.

In the context of the present specification, the term “therapy” and “treatment” includes prevention and prophylaxis, unless there are specific indications to the contrary. The terms “treat”, “therapeutic” and “therapeutically” should be construed accordingly.

In this specification, unless stated otherwise, the term “inhibitor” and “antagonist” mean a compound that by any means, partly or completely, blocks the transduction pathway leading to the production of a response by the ligand.

The term “disorder”, unless stated otherwise, means any condition and disease associated with vanilloid receptor activity.

### Non-Medical Use

In addition to their use in therapeutic medicine, the compounds of the invention, or salts, solvates or solvated salts thereof, are also useful as pharmacological tools in the development and standardisation of in vitro and in vivo test systems for the evaluation of the effects of inhibitors of VR1 related activity in laboratory animals such as cats, dogs, rabbits, monkeys, rats and mice, as part of the search for new therapeutics agents.

### Pharmaceutical Composition

According to one embodiment of the present invention there is provided a pharmaceutical composition comprising as active ingredient a therapeutically effective amount of the compound of formula I, or salts, solvates or solvated salts thereof, in association with one or more pharmaceutically acceptable diluents, excipients and/or inert carriers.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream or for rectal administration e.g. as a suppository.

In general the above compositions may be prepared in a conventional manner using one or more conventional excipients, pharmaceutical acceptable diluents and/or inert carriers. Suitable daily doses of the compounds of formula I in the treatment of a mammal, including man, are approximately 0.01 to 250 mg/kg bodyweight at peroral administration and about 0.001 to 250 mg/kg bodyweight at parenteral administration.

The typical daily dose of the active ingredient varies within a wide range and will depend on various factors such as the relevant indication, severity of the illness being treated, the route of administration, the age, weight and sex of the patient and the particular compound being used, and may be determined by a physician.

### Examples of Pharmaceutical Composition

The following illustrate representative pharmaceutical dosage forms containing a compound of formula I, or salts, solvates or solvated salts thereof, (hereafter compound X) for preventive or therapeutic use in mammals:

| (a): Tablet | mg/tablet |
|---|---|
| Compound X | 100 |
| Lactose | 182.75 |
| Croscarmellose sodium | 12.0 |
| Maize starch paste (5% w/v paste) | 2.25 |
| Magnesium stearate | 3.0 |

| (b): Capsule | mg/capsule |
|---|---|
| Compound X | 10 |
| Lactose | 488.5 |
| Magnesium stearate | 1.5 |

| (c): Injection | (50 mg/ml) | |
|---|---|---|
| Compound X | 5.0% | w/v |
| 1M Sodium hydroxide solution | 15.0% | v/v |
| 0.1M Hydrochloric acid | (to adjust pH to 7.6) | |
| Polyethylene glycol 400 | 4.5% | w/v |

| | |
|---|---|
| Water for injection | up to 100% |

The above compositions may be obtained by conventional procedures well known in the pharmaceutical art.

### Methods of Preparation

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). References and descriptions of other suitable reactions are described in textbooks of organic chemistry, for example, “Advanced Organic Chemistry”, March, 4^{th} ed. McGraw Hill (1992) or, “Organic Synthesis”, Smith, McGraw Hill, (1994). For representative examples of heterocyclic chemistry see for example “Heterocyclic Chemistry”, J. A. Joule, K. Mills, G. F. Smith, 3^{rd} ed. Chapman and Hall (1995), p. 189-224 and “Heterocyclic Chemistry”, T. L. Gilchrist, 2^{nd} ed. Longman Scientific and Technical (1992), p. 248-282.

The term “room temperature” and “ambient temperature” shall mean, unless otherwise specified, a temperature between 16 and 25° C.

One embodiment of the invention relates to processes for the preparation of the compound of formula I according to scheme 1, 2, 3, 4, 5, or 6; wherein R¹ to R⁹, X, n and p are as defined above;

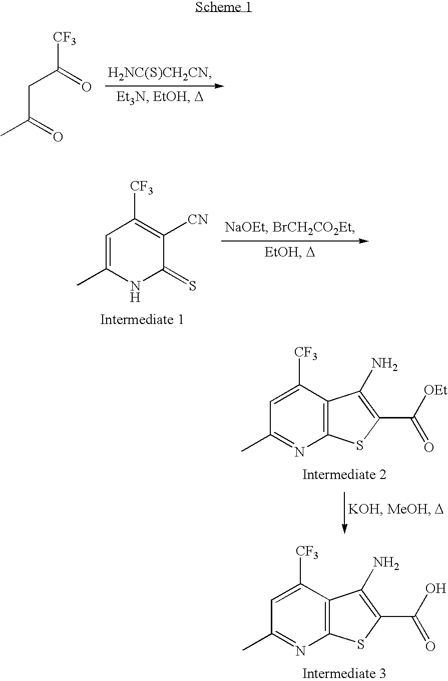

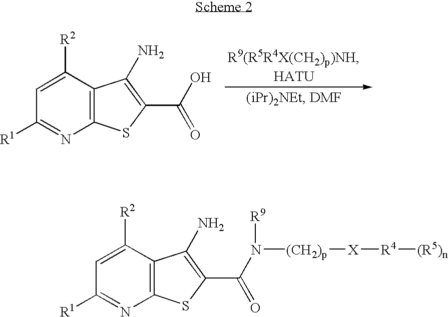

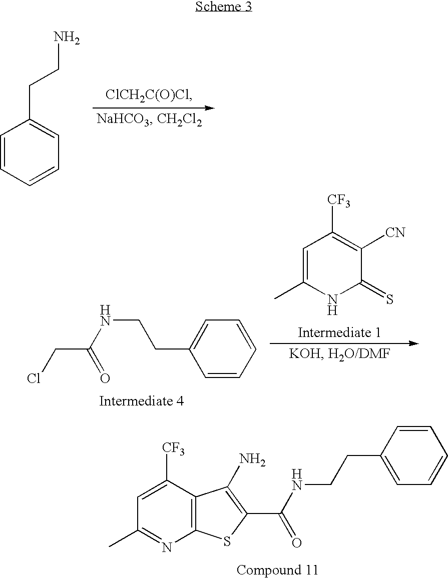

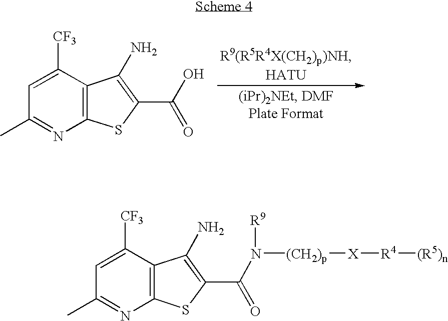

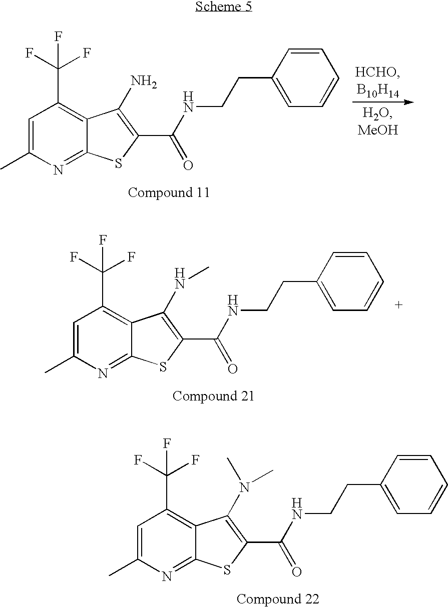

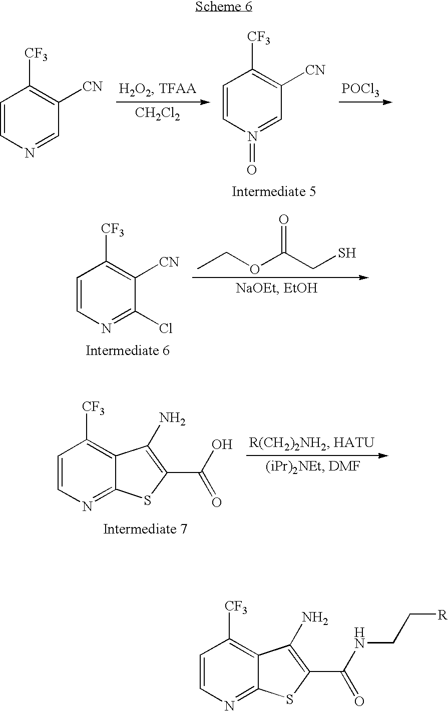

### Intermediates

One embodiment of the invention relates to the compounds
4-(trifluoromethyl)nicotinonitrile 1-oxide,
2-chloro-4-(trifluoromethyl)nicotinonitrile,
3-amino-4-(trifluoromethyl)thieno[2,3-b]pyridine-2-carboxylic acid, and
3-amino-6-methyl-4-(trifluoromethyl)thieno[2,3-b]pyridine-2-carboxylic acid,
   which may be used as intermediates in the preparation of compounds suited for the treatment of VR1 mediated disorders, especially for use as intermediates for the preparation of compounds of formula I.

### EXAMPLES

The invention will now be illustrated by the following non-limiting examples.

### General Methods

The invention will now be illustrated by the following Examples in which, generally:

(i) operations were carried out at ambient or room temperature, i.e. in the range 17 to 25° C. and under an atmosphere of an inert gas such as argon unless otherwise stated;

(ii) evaporations were carried out by rotary evaporation in vacuo and work-up procedures were carried out after removal of residual solids by filtration;

(iii) column chromatography (by the flash procedure) was performed on Silicycle silica gel (grade 230-400 mesh, 60 Å, cat. Numb. R10030B) or obtained from Silicycle, Quebec, Canada or high pressure liquid chromatography (HPLC) was performed on C18 reverse phase silica, for example on a Phenomenex, Luna C-18 100 Å preparative reversed-phase column;

(iv) The ¹H NMR spectra were recorded on Brucker at 400 MHz. The mass spectra were recorded utilising electrospray (LC-MS; LC:Waters 2790, column XTerra MS C₈ 2.5 μm 2.1×30 mm, buffer gradient H₂O+0.1% TFA:CH₃CN+0.04% TFA, MS: micromass ZMD//ammonium acetate buffer) ionisation techniques;

(v) yields, where present, are not necessarily the maximum attainable;

(vi) intermediates were not necessarily fully purified but their structures and purity were assessed by thin layer chromatographic, HPLC and/or NMR analysis

(vii) the following abbreviations have been used:—
HATU O-(7-azabenzotriazol-1-yl)-N,N,N′,N′-tetramethyluronium hexafluorophosphate
HPLC high performance liquid chromatography
LC liquid chromatography
MS mass spectometry
ret. time retention time
TFA trifluoroacetic acid
DMF dimethyflormamide
DIPEA Diisopropylethylamine
NEt₃ Triethylamine

### General Procedure 1 for Amide Formation Between a Carboxylic Acid and Amine:

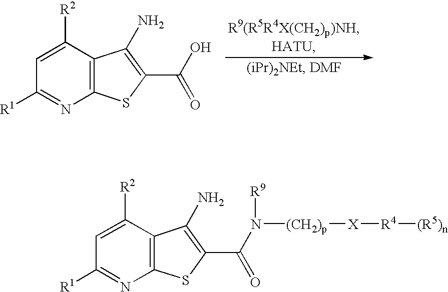

The amine (1 equiv.) was added to a solution of the 3-aminothieno[2,3-b]pyridine-2-carboxylic acid (1 equiv.), HATU (1.1 equiv.) and DIPEA (1.5 equiv.) in DMF (10 mL/mmol of carboxylic acid). The reaction was stirred overnight at room temperature and was then concentrated in vacuo. The residue was redissolved in CH₂Cl₂ and saturated NaHCO₃(aq), and the resulting mixture was loaded onto an Extube® Chem Elut column (Varian). The compound was eluted with four column volumes of CH₂Cl₂. The eluant was concentrated in vacuo, and the crude product was purified by silica gel column chromatography or reverse phase HPLC to provide the title compound.

### General Procedure 2 for Amide Formation Between a Carboxylic Acid and Amine in Plate Format:

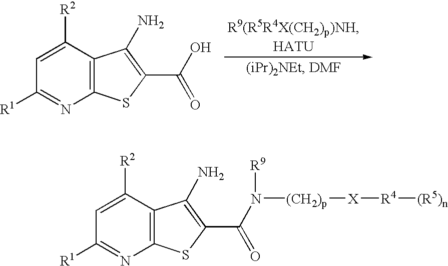

Stock solutions of the 3-aminothieno[2,3-b]pyridine-2-carboxylic acids (0.625 M), amines (0.25 M), HATU (0.55 M), and DIPEA (0.75 M) in DMF were prepared. The solutions of the carboxylic acids were dispensed into 96-well plates (200 μL/well), followed by HATU (250 μL/well), DIPEA (250 μL/well) and the amines (500 μL/well). The 96-well plates were agitated for 2 days, and were then concentrated in vacuo. The residues were redissolved in CH₂Cl₂ and 5% NaOH(aq), mixed, and then filtered through a Unifilter® plate containing Hydromatrix.® The wells were rinsed with additional CH₂Cl₂, and the combined filtrates were concentrated in vacuo. The products were purified by reverse phase HPLC to provide the title compounds. Compounds prepared by this route are listed in Table 1.

### Examples

### Intermediate 1: 6-methyl-2-thioxo-4-(trifluoromethyl)-1,2-dihydropyridine-3-carbonitrile

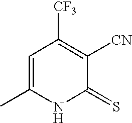

A mixture of 1,1,1-trifluoropentane-2,4-dione (8.159 g, 52.9 mmol), 2-cyano-ethanethioamide (5.302 g, 52.9 mmol) and triethylamine (0.27 mL, 1.9 mmol) was heated in refluxing ethanol (42 mL) for 20 minutes. The reaction was allowed to cool, and the resulting orange solid was transferred to a round bottomed flask using methanol and CH₂Cl₂. The mixture was concentrated in vacuo to provide the title compound, which was used in subsequent steps without further purification. ¹H NMR (400 MHz, DMSO-D₆): δ ppm 2.46 (s, 3H), 7.13 (s, 1H).

### Intermediate 2: ethyl 3-amino-6-methyl-4-(trifluoromethyl)thieno[2,3-b]pyridine-2-carboxylate

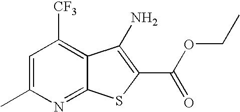

To a mixture of 6-methyl-2-thioxo-4-(trifluoromethyl)-1,2-dihydropyridine-3-carbonitrile (11.5 g, 52.9 mmol) and ethyl bromoacetate (5.9 mL, 53 mmol) in ethanol (235 mL) was added sodium ethoxide (5.40 g, 79 mmol). The reaction was heated to reflux for 2 hours, and additional sodium ethoxide was added, if necessary, until the cyclization was complete as determined by ¹H-NMR. The reaction was concentrated in vacuo, and the residue was taken up in water and CH₂Cl₂. The layers were separated, and the aqueous layer was extracted with additional CH₂Cl₂ (3×). The combined organic phases were dried over Na₂SO₄, filtered, and concentrated in vacuo. The crude product was purified by silica gel chromatography eluting with CH₂Cl₂ to provide the title compound as a yellow solid (14.6 g, 91%). ¹H NMR (400 MHz, CDCl₃) δ ppm 1.39 (t, J=7.1 Hz, 3H), 2.73 (s, 3H), 4.36 (q, J=7.0 Hz, 2H), 6.34 (br s, 2H), 7.41 (s, 1H)

### Intermediate 3: 3-amino-6-methyl-4-(trifluoromethyl)thieno[2,3-b]pyridine-2-carboxylic acid

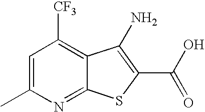

A mixture of ethyl 3-amino-6-methyl-4-(trifluoromethyl)thieno[2,3-b]pyridine-2-carboxylate (14.6 g, 48.0 mmol) and potassium hydroxide (6.73 g, 120 mmol) in 5.5:1 methanol:water (260 mL) was heated at reflux for 4.5 hours. The reaction was concentrated in vacuo, and the residue was taken up in water (90 mL). The pH of the water solution was adjusted to 2 using 1M HCl, and the precipitated yellow solid was collected by filtration. The solid was suspended in water and lyophilized to provide the title compound as a yellow solid (12.5 g, 94%). ¹H NMR (400 MHz, CD₃OD) δ ppm 2.71 (s, 3H), 7.64 (s, 1H)

### Compound 1: 3-amino-6-methyl-N-(3-phenylpropyl)-4-(trifluoromethyl)thieno[2,3-b]pyridine-2-carboxamide

Following General Procedure 1, 3-amino-6-methyl-4-(trifluoromethyl)thieno[2,3-b]pyridine-2-carboxylic acid (0.0769 g, 0.28 mmol), HATU (0.116 g, 0.31 mmol), DIPEA (0.073 mL, 0.42 mmol) and (3-phenylpropyl)amine (0.040 mL, 0.28 mmol) were combined. The title compound was obtained as a yellow gum (0.0839 g, 77%) following purification by reverse phase HPLC (gradient 30-90% CH₃CN in H₂O) and lyophilization from CH₃CN/H₂O. Purity (HPLC): >99%; ¹H-NMR (400 MHz, CDCl₃): δ ppm 1.92-2.02 (m, 2H), 2.68-2.76 (m, 5H), 3.39-3.51 (m, 2H), 5.54 (t, J=5.5 Hz, 1H), 6.48 (br s, 2H), 7.15-7.23 (m, 3H), 7.26-7.33 (m, 2H), 7.44 (s, 1H). MS (ESI) (M+H)⁺=394. Anal. Calcd for C₁₉H₁₈N₃OSF₃+0.2H₂O: C, 57.48; H, 4.67; N, 10.58. Found: C, 57.51; H, 4.40; N, 10.54.

### Compound 2: 3-amino-6-methyl-N-[2-(4-methylphenyl)ethyl]-4-(trifluoromethyl)thieno[2,3-b]pyridine-2-carboxamide

Following General Procedure 1, 3-amino-6-methyl-4-(trifluoromethyl)thieno[2,3-b]pyridine-2-carboxylic acid (0.0751 g, 0.27 mmol), HATU (0.114 g, 0.30 mmol), DIPEA (0.070 mL, 0.40 mmol) and [2-(4-methylphenyl)ethyl]amine (0.54 mL of a 0.5 M DMF solution, 0.27 mmol) were combined. The title compound was obtained as a yellow solid (0.0472 g, 44%) following purification by reverse phase HPLC (gradient 60-100% CH₃CN in H₂O) and lyophilization from CH₃CN/H₂O. Purity (HPLC): >98%; ¹H-NMR (400 MHz, CDCl₃): δ ppm 2.33 (s, 3H), 2.72 (s, 3H), 2.87 (t, J=6.9 Hz, 2H), 3.60-3.69 (m, 2H), 5.53-5.66 (m, 1H), 6.48 (br s, 2H), 7.09-7.17 (m, 4H), 7.44 (s, 1H). MS (ESI) (M+H)⁺=394. Anal. Calcd for C₁₉H₁₈N₃OSF₃+0.1 TFA: C, 56.96; H, 4.51; N, 10.38. Found: C, 57.03; H, 4.50; N, 10.26.

### Compound 3: 3-amino-6-methyl-N-[2-(2-methylphenyl)ethyl]-4-(trifluoromethyl)thieno[2,3-b]pyridine-2-carboxamide

Following General Procedure 1, 3-amino-6-methyl-4-(trifluoromethyl)thieno[2,3-b]pyridine-2-carboxylic acid (0.0751 g, 0.27 mmol), HATU (0.114 g, 0.30 mmol), DIPEA (0.070 mL, 0.40 mmol) and [2-(2-methylphenyl)ethyl]amine (0.54 mL of a 0.5 M DMF solution, 0.27 mmol) were combined. The title compound was obtained as a yellow solid (0.0820 g, 77%) following purification by reverse phase HPLC (gradient 60-100% CH₃CN in H₂O) and lyophilization from CH₃CN/H₂O. Purity (HPLC): >98%; ¹H-NMR (400 MHz, CDCl₃): δ ppm 2.37 (s, 3H), 2.73 (s, 3H), 2.93 (t, J=7.1 Hz, 2H), 3.57-3.71 (m, 2H), 5.62 (t, J=5.4 Hz, 1H), 6.50 (br s, 2H), 7.13-7.22 (m, 4H), 7.44 (s, 1H). MS (ESI) (M+H)⁺=394. Anal. Calcd for C₁₉H₁₈N₃OSF₃: C, 58.01; H, 4.61; N, 10.68. Found: C, 57.79; H, 4.35; N, 10.41.

### Compound 4: 3-amino-6-methyl-N-(2-phenylpropyl)-4-(trifluoromethyl)thieno[2,3-b]pyridine-2-carboxamide

Following General Procedure 1, 3-amino-6-methyl-4-(trifluoromethyl)thieno[2,3-b]pyridine-2-carboxylic acid (0.0751 g, 0.27 mmol), HATU (0.114 g, 0.30 mmol), DIPEA (0.070 mL, 0.40 mmol) and (2-phenylpropyl)amine (0.54 mL of a 0.5 M DMF solution, 0.27 mmol) were combined. The title compound was obtained as a yellow solid (0.0755 g, 71%) following purification by reverse phase HPLC (gradient 50-80% CH₃CN in H₂O) and lyophilization from CH₃CN/H₂O. Purity (HPLC): >96%; ¹H-NMR (400 MHz, CDCl₃): δ ppm 1.35 (d, J=6.8 Hz, 3H), 1.83 (br s, 2H), 2.71 (s, 3H), 2.97-3.12 (m, 1H), 3.35 (ddd, J=13.3, 8.5, 4.9 Hz, 1H), 3.78 (ddd, J=13.3, 7.0, 6.1 Hz, 1H), 5.43 (t, J=5.4 Hz, 1H), 7.18-7.29 (m, 3H), 7.31-7.39 (m, 2H), 7.43 (s, 1H). MS (ESI) (M+H)⁺=394. Anal. Calcd for C₁₉H₁₈N₃OSF₃+0.1 TFA+0.2H₂O: C, 56.46; H, 4.57; N, 10.29. Found: C, 56.35; H, 4.45; N, 10.36.

### Compound 5: 3-amino-N,6-dimethyl-N-(2-phenylethyl)-4-(trifluoromethyl)thieno[2,3-b]pyridine-2-carboxamide

Following General Procedure 1, 3-amino-6-methyl-4-(trifluoromethyl)thieno[2,3-b]pyridine-2-carboxylic acid (0.0751 g, 0.27 mmol), HATU (0.114 g, 0.30 mmol), DIPEA (0.070 mL, 0.40 mmol) and N-methyl-2-phenylethanamine (0.54 mL of a 0.5 M DMF solution, 0.27 mmol) were combined. The title compound was obtained as a yellow solid (0.0748 g, 70%) following purification by reverse phase HPLC (gradient 50-80% CH₃CN in H₂O) and lyophilization from CH₃CN/H₂O. Purity (HPLC): >95%; ¹H-NMR (400 MHz, CDCl₃): δ ppm 2.73 (s, 3H), 2.95-3.03 (m, 2H), 3.14 (s, 3H), 3.76-3.86 (m, 2H), 7.17-7.33 (m, 5H), 7.44 (s, 1H). MS (ESI) (M+H)⁺=394. Anal. Calcd for C₁₉H₁₈N₃OSF₃+0.1 TFA: C, 56.96; H, 4.51; N, 10.38. Found: C, 56.99; H, 4.40; N, 10.78.

### Compound 6: 3-amino-N-[2-(2-methoxyphenyl)ethyl]-6-methyl-4-(trifluoromethyl)thieno[2,3-b]pyridine-2-carboxamide

Following General Procedure 1, 3-amino-6-methyl-4-(trifluoromethyl)thieno[2,3-b]pyridine-2-carboxylic acid (0.0751 g, 0.27 mmol), HATU (0.114 g, 0.30 mmol), DIPEA (0.070 mL, 0.40 mmol) and [2-(2-methoxyphenyl)ethyl]amine (0.54 mL of a 0.5 M DMF solution, 0.27 mmol) were combined. The title compound was obtained as a yellow solid (0.0782 g, 70%) following purification by reverse phase HPLC (gradient 50-80% CH₃CN in H₂O) and lyophilization from CH₃CN/H₂O. Purity (HPLC): >98%; ¹H-NMR (400 MHz, CDCl₃): δ ppm 2.04 (br s, 1H), 2.73 (s, 3H), 2.92-2.98 (m, 2H), 3.61-3.67 (m, 2H), 3.94 (s, 3H), 6.17 (t, J=4.1 Hz, 1H), 6.48 (br s, 1H), 6.87-6.92 (m, 1H), 6.92-6.96 (m, 1H), 7.18 (dd, J=7.4, 1.8 Hz, 1H), 7.22 (dd, J=7.5, 1.7 Hz, 1H), 7.43 (s, 1H). MS (ESI) (M+H)⁺=410. Anal. Calcd for C₁₉H₁₈N₃O₂SF₃+0.2H₂O: C, 55.25; H, 4.49; N, 10.17. Found: C, 55.11; H, 4.30; N, 10.26.

### Compound 7: 3-amino-N-(2,2-diphenylethyl)-6-methyl-4-(trifluoromethyl)thieno[2,3-b]pyridine-2-carboxamide

Following General Procedure 1, 3-amino-6-methyl-4-(trifluoromethyl)thieno[2,3-s b]pyridine-2-carboxylic acid (0.0751 g, 0.27 mmol), HATU (0.114 g, 0.30 mmol), DIPEA (0.070 mL, 0.40 mmol) and (2,2-diphenylethyl)amine (0.54 mL of a 0.5 M DMF solution, 0.27 mmol) were combined. The title compound was obtained as a yellow solid (0.0849 g, 69%) following purification by reverse phase HPLC (gradient 60-100% CH₃CN in H₂O) and lyophilization from CH₃CN/H₂O. Purity (HPLC): >98%; ¹H-NMR (400 MHz, CDCl₃): δ ppm 1.60 (br s, 1H), 2.70 (s, 3H), 4.05 (dd, J=7.9, 5.8 Hz, 2H), 4.27 (t, J=7.9 Hz, 1H), 5.48 (t, J=5.5 Hz, 1H), 6.46 (br s, 1H), 7.20-7.37 (m, 10H), 7.42 (s, 1H). MS (ESI) (M+H)⁺=456. Anal. Calcd for C₂₄H₂₀N₃OSF₃+0.1 TFA: C, 62.25; H, 4.34; N, 9.00. Found: C, 62.44; H, 4.21; N, 8.87.

### Compound 8: 3-amino-N-[2-(3-fluorophenyl)ethyl]-6-methyl-4-(trifluoromethyl)thieno[2,3-b]pyridine-2-carboxamide

Following General Procedure 1, 3-amino-6-methyl-4-(trifluoromethyl)thieno[2,3-b]pyridine-2-carboxylic acid (0.0751 g, 0.27 mmol), HATU (0.114 g, 0.30 mmol), DIPEA (0.070 mL, 0.40 mmol) and [2-(3-fluorophenyl)ethyl]amine (0.54 mL of a 0.5 M DMF solution, 0.27 mmol) were combined. The title compound was obtained as a yellow solid (0.0752 g, 70%) following purification by reverse phase HPLC (gradient 50-80% CH₃CN in H₂O) and lyophilization from CH₃CN/H₂O. Purity (HPLC): >98%; ¹H-NMR (400 MHz, CDCl₃): δ ppm 1.59 (br s, 1H), 2.73 (s, 3H), 2.92 (t, J=7.0 Hz, 2H), 3.67 (q, 2H), 5.59 (t, J=5.6 Hz, 1H), 6.50 (br s, 1H), 6.89-6.98 (m, 2H), 7.01 (d, J=8.0 Hz, 1H), 7.25-7.33 (m, 1H), 7.44 (s, 1H). MS (ESI) (M+H)⁺=398. Anal. Calcd for C₁₈H₁₅N₃OSF₄: C, 54.40; H, 3.80; N, 10.57. Found: C, 54.10; H, 3.64; N, 10.59.

### Compound 9: 3-amino-N-[2-(3,4-dichlorophenyl)ethyl]-6-methyl-4-(trifluoromethyl)thieno[2,3-b]pyridine-2-carboxamide

Following General Procedure 1, 3-amino-6-methyl-4-(trifluoromethyl)thieno[2,3-b]pyridine-2-carboxylic acid (0.0751 g, 0.27 mmol), HATU (0.114 g, 0.30 mmol), DIPEA (0.070 mL, 0.40 mmol) and [2-(3,4-dichlorophenyl)ethyl]amine (0.54 mL of a 0.5 M DMF solution, 0.27 mmol) were combined. The title compound was obtained as a yellow solid (0.0791 g, 65%) following purification by reverse phase HPLC (gradient 60-100% CH₃CN in H₂O) and lyophilization from CH₃CN/H₂O. Purity (HPLC): >98%; ¹H-NMR (400 MHz, CDCl₃): δ ppm 1.58 (br s, 1H), 2.73 (s, 3H), 2.88 (t, J=7.0 Hz, 2H), 3.64 (q, J=6.1 Hz, 2H), 5.60 (t, J=5.8 Hz, 1H), 6.51 (br s, 1H), 7.07 (dd, J=8.1, 2.1 Hz, 1H), 7.34 (d, J=2.1 Hz, 1H), 7.38 (d, J=8.2 Hz, 1H), 7.45 (s, 1H). MS (ESI) (M+H)⁺=448. Anal. Calcd for C₁₈H₁₄N₃OSF₃Cl₂: C, 48.23; H, 3.15; N, 9.37. Found: C, 47.99; H, 2.98; N, 9.30.

### Compound 10: 3-amino-6-methyl-4-(trifluoromethyl)-N-{2-[3-(trifluoromethyl)phenyl]ethyl}thieno[2,3-b]pyridine-2-carboxamide

Following General Procedure 1, 3-amino-6-methyl-4-(trifluoromethyl)thieno[2,3-b]pyridine-2-carboxylic acid (0.0751 g, 0.27 mmol), HATU (0.114 g, 0.30 mmol), DIPEA (0.070 mL, 0.40 mmol) and {2-[3-(trifluoromethyl)phenyl]ethyl}amine (0.54 mL of a 0.5 M DMF solution, 0.27 mmol) were combined. The title compound was obtained as a yellow solid (0.0814 g, 67%) following purification by reverse phase HPLC (gradient 60-100% CH₃CN in H₂O) and lyophilization from CH₃CN/H₂O. Purity (HPLC): >96%; ¹H-NMR (400 MHz, CDCl₃): δ ppm 1.57 (br s, 1H), 2.73 (s, 3H), 2.99 (t, J=7.1 Hz, 2H), 3.63-3.73 (m, 2H), 5.61 (t, J=5.8 Hz, 1H), 6.51 (br s, 1H), 7.40-7.47 (m, 3H), 7.48-7.54 (m, 2H). MS (ESI) (M+H)⁺=448. Anal. Calcd for C₁₉H₁₅N₃OSF₆+0.1 TFA: C, 50.26; H, 3.32; N, 9.16. Found: C, 50.17; H, 3.17; N, 9.18.

### Intermediate 4: 2-chloro-N-(2-phenylethyl)acetamide

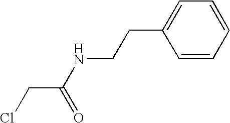

Chloroacetyl chloride (1.95 mL, 24.5 mmol) was added dropwise to a mixture of (2-phenylethyl)amine (2.476 g, 20.4 mmol) and sodium bicarbonate (2.16 g, 25.7 mmol) in CH₂Cl₂ (20 mL) maintained at 0° C. The reaction was stirred for 2.5 hours at 10° C., and was then cooled back down to 0° C. and quenched by the addition of water (10 mL). The layers were separated, and the organic phase was washed successively with 10% HCl_{(aq)} and brine. The organic phase was then dried over Na₂SO₄, filtered, and concentrated in vacuo to provide the title compound (4.13 g, quantitative), which was used in subsequent steps without further purification. ¹H NMR (400 MHz, CDCl₃): δ ppm 2.86 (t, J=7.0 Hz, 2H), 3.51-3.64 (m, 2H), 4.04 (s, 2H), 6.63 (br s, 1H), 7.18-7.28 (m, 3H), 7.30-7.37 (m, 2H).

### Compound 11: 3-amino-6-methyl-N-(2-phenylethyl)-4-(trifluoromethyl)thieno[2,3-b]pyridine-2-carboxamide

To a solution of 6-methyl-2-thioxo-4-(trifluoromethyl)-1,2-dihydropyridine-3-carbonitrile (0.259 g, 1.19 mmol) in DMF (2 mL) was added 2-chloro-N-(2-phenylethyl)acetamide (0.234 g, 1.19 mmol) in portions and a solution of 15% sodium hydroxide in water (0.65 mL, 1.8 mmol) dropwise. The resulting mixture was stirred at room temperature for 3.5 hours, and was then diluted with water (10 mL) and CH₂Cl₂ (20 mL). The layers were separated, and the aqueous layer was extracted with additional CH₂Cl₂ (3×). The combined organic phases were washed with brine (2×), and then dried over Na₂SO₄, filtered, and concentrated in vacuo. The crude product was purified by silica gel chromatography eluting with 5:1 CH₂Cl₂:EtOAc, followed by a second purification eluting with 3:1 hexanes:EtOAc, to provide the title compound as a yellow solid (0.211 g, 47%). Purity (HPLC): >99%; ¹H-NMR (400 MHz, CDCl₃): δ ppm 2.73 (s, 3H), 2.93 (t, J=6.9 Hz, 2H), 3.65-3.72 (m, 2H), 5.61 (t, J=5.7 Hz, 1H), 6.51 (br s, 2H), 7.22-7.30 (m, 3H), 7.30-7.38 (m, 2H), 7.45 (s, 1H). MS (ESI) (M+H)⁺=380. Anal. Calcd for C₁₈H₁₆N₃OSF₃: C, 56.98; H, 4.25; N, 11.08. Found: C, 56.64; H, 4.21; N, 10.93.

### Compound 12: 3-amino-6-methyl-N-[(2S)-2-phenylpropyl]-4-(trifluoromethyl)thieno[2,3-b]pyridine-2-carboxamide

Following General Procedure 1, 3-amino-6-methyl-4-(trifluoromethyl)thieno[2,3-h]pyridine-2-carboxylic acid (0.200 g, 0.72 mmol), HATU (0.303 g, 0.78 mmol), DIPEA (0.19 mL, 1.08 mmol), and (S)-(−)-β-methylphenethylamine (155 μL, 1.08 mmol) were combined. The title compound was obtained as a yellow solid (0.248 g, 87%) following purification by reverse phase HPLC (gradient 30-90% CH₃CN in H₂O) and lyophilization from CH₃CN/H₂O. Purity (HPLC): >99%; Chiral Purity (HPLC): >99%; ¹H NMR (400 MHZ, CDCl₃): δ ppm 1.36 (d, J=7.03 Hz, 3H), 2.72 (s, 3H), 3.00-3.18 (m, 1H), 3.32-3.41 (m, 1H), 3.75-3.84 (m, 1H), 5.43 (t, J=5.66 Hz, 1H), 6.47 (s, 2H), 7.24-7.29 (m, 3H), 7.33-7.39 (m, 2H), 7.44 (s, 1H). MS (ESI) (M+H)⁺=394. Anal. Calcd for C₁₉H₁₈F₃N₃OS+0.15H₂O: C, 57.61; H, 4.66; N, 10.61. Found: C, 57.48; H, 4.48; N, 10.45. Optical Rotation: [α]^{D}₁₈=−76.9° (c=0.963, MeOH).

### Compound 13: 3-amino-6-methyl-N-[(2R)-2-phenylpropyl]-4-(trifluoromethyl)thieno[2,3-b]pyridine-2-carboxamide

Following General Procedure 1, 3-amino-6-methyl-4-(trifluoromethyl)thieno[2,3-b]pyridine-2-carboxylic acid (0.150 g, 0.54 mmol), HATU (0.310 g, 0.81 mmol), DIPEA (0.24 mL, 1.38 mmol), and (R)-(+)-β-methylphenethylamine (110 μL, 0.77 mmol) were combined. The title compound was obtained as a yellow solid (0.143 g, 67%) following purification by column chromatography (25% ethyl acetate in hexanes). Purity (HPLC): >99%; Chiral Purity (HPLC): >99%; ¹H NMR (400 MHZ, CD₃OD): δ ppm 1.28 (d, J=7.0 Hz, 3H), 2.99 (s, 3H), 3.03-3.14 (m, 1H), 3.45-3.51 (m, 2H), 7.13-7.19 (m, 1H), 7.22-7.30 (m, 4H); 7.64 (s, 1H). MS (ESI) (M+H)⁺=394. Anal. Calcd for C₁₉H₁₈F₃N₃OS×0.2HCl: C, 56.95; H, 4.58; N, 10.49. Found: C, 57.06; H, 4.47; N, 10.67.

### Compound 14: 3-amino-N-[(2R)-2-hydroxy-2-phenylethyl]-6-methyl-4-(trifluoromethyl)thieno[2,3-b]pyridine-2-carboxamide

Following General Procedure 1, 3-amino-6-methyl-4-(trifluoromethyl)thieno[2,3-b]pyridine-2-carboxylic acid (0.0751 g, 0.27 mmol), HATU (0.114 g, 0.30 mmol), DIPEA (0.14 mL, 0.80 mmol), and (1R)-2-amino-1-phenylethanol (0.0370 g, 0.27 mmol) were combined. The title compound was obtained as a yellow solid (0.0638 g, 59%) following purification by reverse phase HPLC (gradient 50-80% CH₃CN in H₂O) and lyophilization from CH₃CN/H₂O. Purity (HPLC): >99%; ¹H NMR (400 MHZ, CDCl₃): δ ppm 2.73 (s, 3H), 3.28 (d, J=3.5 Hz, 1H), 3.52 (ddd, J=14.1, 8.0, 5.1 Hz, 1H), 3.85 (ddd, J=14.2, 6.9, 3.3 Hz, 1H), 4.96 (ddd, J=7.6, 3.7, 3.4 Hz, 1H), 5.99 (t, J=5.7 Hz, 1H), 6.53 (s, 2H), 7.27-7.34 (m, 1H), 7.34-7.44 (m, 4H), 7.45 (s, 1H). MS (ESI) (M+H)⁺=396.

### Compound 15: 3-amino-N-[(2S)-2-hydroxy-2-phenylethyl]-6-methyl-4-(trifluoromethyl)thieno[2,3-b]pyridine-2-carboxamide

Following General Procedure 1, 3-amino-6-methyl-4-(trifluoromethyl)thieno[2,3-b]pyridine-2-carboxylic acid (0.0751 g, 0.27 mmol), HATU (0.114 g, 0.30 mmol), DIPEA (0.14 mL, 0.80 mmol), and (1S)-2-amino-1-phenylethanol (0.0370 g, 0.27 mmol) were combined. The title compound was obtained as a yellow solid (0.0631 g, 59%) following purification by reverse phase HPLC (gradient 50-80% C₃CN in H₂O) and lyophilization from CH₃CN/H₂O. Purity (HPLC): >99%; ¹H NMR (400 MHZ, CDCl₃): δ ppm 2.73 (s, 3H), 3.27 (d, J=3.3 Hz, 1H), 3.53 (ddd, J=14.1, 8.0, 5.1 Hz, 1H), 3.85 (ddd, J=14.2, 6.8, 3.2 Hz, 1H), 4.96 (ddd, J=7.6, 3.7, 3.4 Hz, 1H), 5.98 (t, J=6.1 Hz, 1H), 6.53 (s, 2H), 7.28-7.33 (m, 1H), 7.34-7.44 (m, 4H), 7.45 (s, 1H). MS (ESI) (M+H)⁺=396. Anal. Calcd for C₁₈H₁₆F₃N₃O₂S+0.1H₂O: C, 54.43; H, 4.11; N, 10.58. Found: C, 54.43; H, 3.81; N, 10.29.

### Compound 16: 3-amino-N-(2-hydroxy-2-phenylpropyl)-6-methyl-4-(trifluoromethyl)thieno[2,3-b]pyridine-2-carboxamide

Following a modified version of General Procedure 1 employing additional DIPEA, 3-amino-6-methyl-4-(trifluoromethyl)thieno[2,3-b]pyridine-2-carboxylic acid (0.101 g, 0.37 mmol), HATU (0.153 g, 0.40 mmol), DIPEA (0.19 mL, 1.1 mmol), and 1-amino-2-phenylpropan-2-ol hydrochloride (0.0685 g, 0.37 mmol) were combined. The title compound was obtained as a yellow solid (0.125 g, 84%) following purification by column chromatography (3:1 CH₂Cl₂:EtOAc). Purity (HPLC): >99%; ¹H NMR (400 MHZ, CDCl-₃): δ ppm 1.62 (s, 3H), 2.70 (s, 3H), 3.44 (s, 1H), 3.55 (dd, J=14.0, 5.2 Hz, 1H), 3.88 (dd, J=14.1, 7.0 Hz, 1H), 5.84 (t, J=5.8 Hz, 1H), 6.48 (s, 2H), 7.23-7.30 (m, 1H), 7.32-7.39 (m, 2H), 7.42 (s, 1H), 7.46-7.54 (m, 2H). MS (ESI) (M+H)⁺=410. Anal. Calcd for C₁₉H₁₈F₃N₃O₂S+0.2H₂O: C, 55.25; H, 4.49; N, 10.17. Found: C, 55.24; H, 4.38; N, 10.50.

### Compound 17: 3-amino-N-[2-(2-furyl)ethyl]-6-methyl-4-(trifluoromethyl)thieno[2,3-b]pyridine-2-carboxamide

Following General Procedure 1, 3-amino-6-methyl-4-(trifluoromethyl)thieno[2,3-b]pyridine-2-carboxylic acid (0.150 g, 0.54 mmol), HATU (0.227 g, 0.60 mmol), DIPEA (0.14 mL, 0.81 mmol), and 2-furan-2-yl-ethylamine (167 mg, 0.81 mmol) were combined. The title compound was obtained as a yellow solid (0.090 g, 45%) following purification by reverse phase HPLC (gradient 30-90% CH₃CN in H₂O) and lyophilization from CH₃CN/H₂O. Purity (HPLC): >99%; ¹H NMR (400 MHZ, CDCl₃): δ ppm 2.74 (s, 3H) 2.96 (t, J=6.54 Hz, 2H) 3.70 (q, J=6.58 Hz, 2H) 5.80 (t, J=5.37 Hz, 1H) 6.13 (dd, J=3.32, 0.78 Hz, 1H) 6.32 (dd, J=3.12, 1.76 Hz, 1H) 6.50 (s, 2H) 7.37 (dd, J=1.86, 0.88 Hz, 1H) 7.45 (s, 1H). MS (ESI) (M+H)⁺=370. Anal. Calcd for C₁₆H₁₄F₃N₃O₂S: C, 52.03; H, 3.82; N, 11.38. Found: C, 51.80; H, 3.64; N, 11.63.

### Compound 18: 3-amino-N-[2-(4-fluorophenyl)ethyl]-6-methyl-4-(trifluoromethyl)thieno[2,3-b]pyridine-2-carboxamide

Following General Procedure 1, 3-amino-6-methyl-4-(trifluoromethyl)thieno[2,3-b]pyridine-2-carboxylic acid (0.150 g, 0.54 mmol), HATU (0.227 g, 0.60 mmol), DIPEA (0.14 mL, 0.81 mmol), and 4-fluorophenethylamine (106 μL, 0.81 mmol) were combined. The title compound was obtained as a yellow solid (0.100 g, 45%) following purification by reverse phase HPLC (gradient 30-90% CH₃CN in H₂O) and lyophilization from CH—₃CN/H₂O. Purity (HPLC): >99%; ¹H NMR (400 MHZ, CDCl₃): δ ppm 2.74 (s, 3H), 2.90 (t, J=6.93 Hz, 2H), 3.65 (q, J=6.05 Hz, 2H), 5.59 (t, J=5.86 Hz, 1H), 6.51 (s, 2H), 6.99-7.06 (m, 2H), 7.17-7.23 (m, 2H), 7.45 (s, 1H). MS (ESI) (M+H)⁺=398. Anal. Calcd for C₁₆H₁₄F₃N₃O₂S: C, 52.03; H, 3.82; N, 11.38. Found: C, 51.80; H, 3.64; N, 11.63.

### Compound 19: 3-amino-N-(2-cyclohexylethyl)-6-methyl-4-(trifluoromethyl)thieno[2,3-b]pyridine-2-carboxamide

Following a modified version of General Procedure 1 employing additional DIPEA, 3-amino-6-methyl-4-(trifluoromethyl)thieno[2,3-b]pyridine-2-carboxylic acid (0.0751 g, 0.27 mmol), HATU (0.114 g, 0.30 mmol), DIPEA (0.14 mL, 0.80 mmol) and 2-cyclohexylethanamine hydrochloride (0.0442 g, 0.27 mmol) were combined. The title compound was obtained as a yellow solid (0.0753 g, 72%) following purification by reverse phase HPLC (gradient 60-100% CH₃CN in H₂O) and lyophilization from CH₃CN/H₂O. Purity (HPLC): >99%; ¹H-NMR (400 MHz, CDCl₃): δ ppm 0.87-1.03 (m, 2H), 1.11-1.26 (m, 3H), 1.27-1.43 (m, 1H), 1.47-1.54 (m, 2H), 1.60-1.82 (m, 5H), 2.73 (s, 3H), 3.40-3.50 (m, 2H), 5.51 (t, J=5.5 Hz, 1H), 6.48 (s, 2H), 7.44 (s, 1H). MS (ESI) (M+H)⁺=386. Anal. Calcd for C₁₈H₂₂N₃OSF₃: C, 56.09; H, 5.75; N, 10.90. Found: C, 55.92; H, 5.68; N, 10.67.

### Compound 20: 3-amino-6-methyl-N-(trans-4-methylcyclohexyl)-4-(trifluoromethyl)thieno[2,3-b]pyridine-2-carboxamide

Following General Procedure 1, 3-amino-6-methyl-4-(trifluoromethyl)thieno[2,3-b]pyridine-2-carboxylic acid (0.150 g, 0.54 mmol), HATU (0.310 g, 0.81 mmol), DIPEA (0.24 mL, 1.38 mmol), and trans-4-methyl-cyclohexylamine HCl (0.12 g, 0.80 mmol) were combined. The title compound was obtained as a yellow solid (0.072 g, 36%) following purification by column chromatography (30% ethyl acetate in hexanes). Purity (HPLC): >99%; ¹H-NMR (400 MHz, CD₃OD): δ ppm 0.90 (d, J=6.4 Hz, 3H), 0.97-1.15 (m, 2H), 1.26-1.47 (m, 3H), 1.67-1.82 (m, 2H), 1.84-1.95 (m, 2H), 2.69 (s, 3H), 3.71-3.86 (m, 1H), 7.64 (s, 1H). MS (ESI) (M+H)⁺=372. Anal. Calcd for C₁₇H₂₀F₃N₃OS×0.1H₂O×0.1HCl: C, 54.18; H, 5.43; N, 11.15. Found: C, 54.32; H, 5.36; N, 11.00.

### Compound 21: 6-methyl-3-(methylamino)-N-(2-phenylethyl)-4-(trifluoromethyl)thieno[2,3-b]pyridine-2-carboxamide

To a solution of Compound 11 (0.120 g, 0.31 mmol) in methanol (5 mL) was added formaldehyde (37% in water, 70 μL, 0.95 mmol). The reaction was stirred overnight at room temperature. The next day, decaborane was added and the reaction was stirred for 2 hours and then concentrated in vacuo. The residue was taken up in dichloromethane and washed with 2 M NaOH. The aqueous layer was extracted with two portions of dichloromethane and the combined organic phases were dried over MgSO₄, filtered and concentrated to give a 1:1 mixture of Compound 21 and Compound 22. The compounds were separated by reverse phase HPLC (40-90% CH₃CN in H₂O). The title compound was obtained as a yellow gum (0.049 g, 40%) following lyophilization from CH₃CN/H₂O. Purity (HPLC): >94%; ¹H NMR (400 MHZ, CDCl₃): δ ppm 2.50 (s, 3H), 2.74 (s, 3H), 2.99 (t, J=6.93 Hz, 2H), 3.78-3.87 (m, 2H), 7.22-7.38 (m, 5H), 7.47 (s, 1H), 9.07 (t, J=5.47 Hz, 1H). MS (ESI) (M+H)⁺=394. Anal. Calcd for C₁₉H₁₈F₃N₃OS+0.35 TFA has C, 54.60; H, 4.27; N, 9.70. Found: C, 54.66; H, 4.14; N, 9.56.

### Compound 22: 3-(dimethylamino)-6-methyl-N-(2-phenylethyl)-4-(trifluoromethyl)thieno[2,3-b]pyridine-2-carboxamide

Isolated from the reaction mixture of Compound 21, the title compound was obtained as a yellow solid (0.051 g, 40%) following purification by reverse phase HPLC (40-90% CH₃CN in H₂O) and lyophilization from CH₃CN/H₂O. Purity (HPLC): >99%; ¹H NMR (400 MHZ, CDCl₃): δ ppm 2.68 (s, 6H), 2.72 (s, 3H), 2.98 (t, J=6.84 Hz, 2H), 3.81 (q, J=6.64 Hz, 2H), 6.82 (br s, 1H), 7.22-7.29 (m, 3H), 7.31-7.37 (m, 2H), 7.49 (s, 1H). MS (ESI) (M+H)⁺=408. Anal. Calcd for C₂₀H₂₀F₃N₃OS has C, 58.96; H, 4.95; N, 10.31. Found: C, 58.78; H, 4.99; N, 10.54.

### Intermediate 5: 4-(trifluoromethyl)nicotinonitrile 1-oxide

4-(Trifluoromethyl)nicotinonitrile (10.0 g, 58.1 mmol) was dissolved in dichloromethane (400 mL) and 30% hydrogen peroxide (11.9 mL, 116 mmol) was added. The solution was cooled to 0° C. and trifluoroacetic anhydride (16.4 mL, 116 mmol) was slowly added via a dropping funnel. The reaction was warmed to 40° C. and stirred overnight. After cooling to room temperature, saturated aqueous Na₂S₂O₃ was added and the solution was poured into a separatory funnel containing 1 M HCl. The layers were separated and the organic layer was washed with saturated aqueous sodium bicarbonate, dried over Na₂SO₄, filtered and concentrated to give the title compound of an off-white solid (10.5 g, 96%). ¹H NMR (400 MHz, CDCl₃): δ ppm 7.65 (d, J=7.03 Hz, 1H), 8.37-8.40 (m, 1H), 8.47-8.49 (m, 1H).

### Intermediate 6: 2-chloro-4-(trifluoromethyl)nicotinonitrile

A mixture of 4-(trifluoromethyl)nicotinonitrile 1-oxide (10.5 g, 55.8 mmol) and POCl₃ (51 mL, 558 mmol) was heated at 110° C. for 5 hours. After evaporation of excess POCl₃, the residue was taken up in dichloromethane and washed successively with 5% K₂CO₃ and water. The organic phase was then dried over Na₂SO₄, filtered and concentrated to give a mixture of the title compound and 6-chloro-4-(trifluoromethyl)nicotinonitrile. ¹H NMR analysis of the crude material showed that the title compound was the major isomer (7:3 2-chloro:6-chloro). The isomers were separated by flash chromatography. The 6-chloro isomer was eluted first with 9:1 hexanes:Et₃N. Eluting with dichloromethane gave the title compound as an orange oil (4.50 g, 38%). ¹H NMR (400 MHz, CDCl₃): δ ppm 7.67 (d, J=5.08 Hz, 1H), 8.81 (d, J=5.08 Hz, 1H).

### Intermediate 7: 3-amino-4-(trifluoromethyl)thieno[2,3-b]pyridine-2-carboxylic acid

A solution of 2-chloro-4-(trifluoromethyl)nicotinonitrile (2.00 g, 9.68 mmol) in ethanol (10 mL) was added to a stirred solution of ethyl 2-mercaptoacetate and sodium ethoxide in ethanol (10 mL). The reaction was heated to reflux for 5 hours, and additional sodium ethoxide was added, if necessary until the cyclization was complete as determined by ¹H NMR. The reaction mixture was poured into a flask containing ice/H₂O and was acidified with 1 M HCl to pH 2. The resulting solid was collected by vacuum filtration to give the title compound as a yellow solid (2.10 g 83%). ¹H NMR (400 MHz, CD₃OD): δ ppm (d, J=4.88 Hz, 1H), 8.83 (d, J=4.88 Hz, 1H).

### Compound 23: 3-amino-N-(2-phenylethyl)-4-(trifluoromethyl)thieno[2,3-b]pyridine-2-carboxamide

Following General Procedure 1, 3-amino-4-(trifluoromethyl)thieno[2,3-b]pyridine-2-carboxylic acid (0.030 g, 0.11 mmol), HATU (0.048 g, 0.13 mmol), DIPEA (28 μL, 0.16 mmol), and (2-phenylethyl)amine (13 μL, 0.16 mmol) were combined. The title compound was obtained as a yellow solid (27.7 mg, 69%) following purification by reverse phase HPLC (gradient 20-90% CH₃CN in H₂O) and lyophilization from CH₃CN/H₂O. Purity (HPLC): >99%; ¹H NMR (400 MHZ, CDCl₃): δ ppm 2.94 (t, J=6.93 Hz, 2H), 3.66-3.74 (m, 2H), 5.65 (t, J=4.69 Hz, 1H), 6.53 (s, 2H) 7.22-7.30 (m, 3H), 7.31-7.39 (m, 2H), 7.59 (d, J=4.88 Hz, 1H), 8.77 (d, J=4.69 Hz, 1H). MS (ESI) (M+H)⁺=366. Anal. Calcd for C₁₇H₁₄F₃N₃OS×0.35 TFA: C, 52.46; H, 3.57; N, 10.37. Found: C, 52.59; H, 3.43; N, 10.47.

### Compound 24: 3-amino-N-[2-(4-methylphenyl)ethyl]-4-(trifluoromethyl)thieno[2,3-b]pyridine-2-carboxamide

Following General Procedure 1, 3-amino-4-(trifluoromethyl)thieno[2,3-b]pyridine-2-carboxylic acid (0.030 g, 0.11 mmol), HATU (0.048 g, 0.13 mmol), DIPEA (28 μL, 0.16 mmol), and [2-(4-methylphenyl)ethyl]amine (14 μL, 0.16 mmol) were combined. The title compound was obtained as a yellow solid (30.0 mg, 72%) following purification by reverse phase HPLC (gradient 20-90% CH₃CN in H₂O) and lyophilization from CH₃CN/H—₂O. Purity (HPLC): >99%; ¹H NMR (400 MHZ, CDCl₃): δ ppm 2.34 (s, 3H), 2.89 (t, J=6.93 Hz, 2H), 3.63-3.70 (m, 2H), 5.64 (s, 1H), 6.52 (s, 2H), 7.11-7.18 (m, 4H), 7.59 (d, J=4.88 Hz, 1H), 8.77 (d, J=4.69 Hz, 1H). MS (ESI) (M+H)⁺=380. Anal. Calcd for C₁₈H₁₆F₃N₃OS+0.05H2O+0.1 TFA: C, 55.81; H, 4.17; N, 10.73. Found: C, 55.40; H, 3.75; N, 11.04.

### Compound 25: 3-amino-N-[2-(3-fluorophenyl)ethyl]-4-(trifluoromethyl)thieno[2,3-b]pyridine-2-carboxamide

Following General Procedure 1, 3-amino-4-(trifluoromethyl)thieno[2,3-b]pyridine-2-carboxylic acid (0.030 g, 0.11 mmol), HATU (0.048 g, 0.13 mmol), DIPEA (28 μL, 0.16 mmol), and [2-(3-fluorophenyl)ethyl]amine (14 μL, 0.16 mmol) were combined. The title compound was obtained as a yellow solid (23.7 mg, 56%) following purification by reverse phase HPLC (gradient 20-90% CH₃CN in H₂O) and lyophilization from CH₃CN/H—₂O. Purity (HPLC): >99%; ¹H NMR (400 MHZ, CDCl₃): δ ppm 2.94 (t, J=7.03 Hz, 2H), 3.66-3.73 (m, 2H), 5.65 (t, J=5.08 Hz, 1H), 6.54 (s, 2H), 6.92-6.99 (m, 2H), 7.03 (d, J=7.62 Hz, 1H), 7.27-7.34 (m, 1H), 7.60 (d, J=4.88 Hz, 1H), 8.77 (d, J=4.69 Hz, 1H). MS (ESI) (M+H)⁺=384. Anal. Calcd for C₁₇H₁₃F₄N₃OS+0.1H₂O+0.25 TFA: C, 51.81; H, 3.28; N, 10.16. Found: C, 51.12; H, 3.11; N, 9.81.

TABLE 1
Compounds prepared according to general General Procedure 2.

| IUPAC Name | Retention Time | MH+ |
|---|---|---|
| 3-amino-6-methyl-N-{2-[3-(methyloxy)phenyl]ethyl}-4- | 1.78 | 410.09 |
| (trifluoromethyl)thieno[2,3-b]pyridine-2-carboxamide | | |
| 3-amino-6-methyl-N-[2-(2-thienyl)ethyl]-4-(trifluoro- | 1.78 | 386.06 |
| methyl)thieno[2,3-b]pyridine-2-carboxamide | | |
| 3-amino-N-[2-(2,6-dichlorophenyl)ethyl]-6-methyl-4- | 1.96 | 448.03 |
| (trifluoromethyl)thieno[2,3-b]pyridine-2-carboxamide | | |
| 3-amino-N-[2-(2-fluorophenyl)ethyl]-6-methyl-4- | 1.83 | 398.09 |
| (trifluoromethyl)thieno[2,3-b]pyridine-2-carboxamide | | |
| 3-amino-6-methyl-N-[2-(phenyloxy)ethyl]-4-(trifluoro- | 1.79 | 396.08 |
| methyl)thieno[2,3-b]pyridine-2-carboxamide | | |
| 3-amino-N-(2,3-dihydro-1,4-benzodioxin-2-ylmethyl)-6- | 1.83 | 424.09 |
| methyl-4-(trifluoromethyl)thieno[2,3-b]pyridine-2-carboxamide | | |
| 3-amino-N-{2-[4-(ethyloxy)phenyl]ethyl}-6-methyl-4- | 1.86 | 424.13 |
| (trifluoromethyl)thieno[2,3-b]pyridine-2-carboxamide | | |
| 3-amino-6-methyl-N-(4-methylcyclohexyl)-4-(trifluoro- | 1.91 | 372.16 |
| methyl)thieno[2,3-b]pyridine-2-carboxamide | | |
| 3-amino-6-methyl-N-{2-[2-(phenyloxy)phenyl]ethyl}-4- | 2.06 | 472.14 |
| (trifluoromethyl)thieno[2,3-b]pyridine-2-carboxamide | | |
| 3-amino-6-methyl-N-{[5-methyl-2-(trifluoromethyl)-3- | 1.93 | 438.06 |
| furanyl]methyl}-4-(trifluoromethyl)thieno[2,3-b]pyridine-2- | | |
| carboxamide | | |
| 1,1-dimethylethyl 4-({[3-amino-6-methyl-4-(trifluoro- | 1.78 | 459.21 |
| methyl)thieno[2,3-b]pyridin-2-yl]carbonyl}amino)-1- | | |
| piperidinecarboxylate | | |
| 3-amino-N-{[3-fluoro-5-(trifluoromethyl)phenyl]methyl}-6- | 1.91 | 452.06 |
| methyl-4-(trifluoromethyl)thieno[2,3-b]pyridine-2-carboxamide | | |
| 3-amino-6-methyl-4-(trifluoromethyl)-N-{[3-(trifluoro- | 1.89 | 434.07 |
| methyl)phenyl]methyl}thieno[2,3-b]pyridine-2-carboxamide | | |
| 3-amino-N-(3,3-dimethylbutyl)-6-methyl-4-(trifluoro- | 1.86 | 360.12 |
| methyl)thieno[2,3-b]pyridine-2-carboxamide | | |
| 3-amino-6-methyl-4-(trifluoromethyl)-N-({3-[(trifluoro- | 1.93 | 450.05 |
| methyl)oxy]phenyl}methyl)thieno[2,3-b]pyridine-2-carboxamide | | |
| 3-amino-N-{2-[4-(1,1-dimethylethyl)phenyl]ethyl}-6- | 2.08 | 436.18 |
| methyl-4-(trifluoromethyl)thieno[2,3-b]pyridine-2-carboxamide | | |
| 3-amino-6-methyl-N-{3-[methyl(phenyl)amino]propyl}-4- | 1.29 | 423.2 |
| (trifluoromethyl)thieno[2,3-b]pyridine-2-carboxamide | | |
| 3-amino-N-[(3,5-dimethylphenyl)methyl]-6-methyl-4- | 1.91 | 394.11 |
| (trifluoromethyl)thieno[2,3-b]pyridine-2-carboxamide | | |
| 3-amino-N-(cyclohexylmethyl)-6-methyl-4-(trifluoro- | 1.89 | 372.13 |
| methyl)thieno[2,3-b]pyridine-2-carboxamide | | |
| 3-amino-N-butyl-6-methyl-4-(trifluoromethyl)thieno[2,3- | 1.66 | 332.08 |
| b]pyridine-2-carboxamide | | |
| 3-amino-N-[2-(2,4-dichlorophenyl)ethyl]-6-methyl-4- | 2.01 | 448.03 |
| (trifluoromethyl)thieno[2,3-b]pyridine-2-carboxamide | | |
| 3-amino-N-cyclohexyl-6-methyl-4-(trifluoro- | 1.79 | 358.13 |
| methyl)thieno[2,3-b]pyridine-2-carboxamide | | |
| 3-amino-N-[(5-fluoro-2-methylphenyl)methyl]-6-methyl-4- | 1.84 | 398.09 |
| (trifluoromethyl)thieno[2,3-b]pyridine-2-carboxamide | | |
| 3-amino-N-[1-(4-fluorophenyl)ethyl]-6-methyl-4- | 1.81 | 398.15 |
| (trifluoromethyl)thieno[2,3-b]pyridine-2-carboxamide | | |
| 3-amino-6-methyl-N-(2-methylpropyl)-4-(trifluoro- | 1.64 | 332.09 |
| methyl)thieno[2,3-b]pyridine-2-carboxamide | | |
| 3-amino-N-[(6-fluoro-4H-1,3-benzodioxin-8-yl)methyl]-6- | 1.78 | 442.09 |
| methyl-4-(trifluoromethyl)thieno[2,3-b]pyridine-2-carboxamide | | |
| 3-amino-N,6-dimethyl-4-(trifluoromethyl)-N-{[3- | 1.93 | 448.08 |
| (trifluoromethyl)phenyl]methyl}thieno[2,3-b]pyridine-2- | | |
| carboxamide | | |
| 3-amino-N-(2,3-dihydro-1-benzofuran-5-ylmethyl)-6- | 1.74 | 408.12 |
| methyl-4-(trifluoromethyl)thieno[2,3-b]pyridine-2-carboxamide | | |
| 3-amino-6-methyl-N-[2-(2-pyridinyl)ethyl]-4-(trifluoro- | 1.11 | 381.08 |
| methyl)thieno[2,3-b]pyridine-2-carboxamide | | |
| 3-amino-6-methyl-N-[2-(4-pyridinyl)ethyl]-4-(trifluoro- | 1.12 | 381.13 |
| methyl)thieno[2,3-b]pyridine-2-carboxamide | | |

### Pharmacology

1. hVR1 FLIPR (Fluorometric Image Plate Reader) Screening Assay

Transfected CHO cells, stably expressing hVR1 (15,000 cells/well) are seeded in 50 ul media in a black clear bottom 384 plate (Greiner) and grown in a humidified incubator (37° C., 2% CO₂), 24-30 hours prior to experiment.

Subsequently, the media is removed from the cell plate by inversion and 2 μM Fluo-4 is added using a multidrop (Labsystems). Following the 40 minutes dye incubation in the dark at 37° C. and 2% CO₂, the extracellular dye present is washed away using an EMBLA (Scatron), leaving the cells in 40 ul of assay buffer (1×HBSS, 10 mM D-Glucose, 1 mM CaCl₂, 10 mM HEPES, 10×7.5% NaHCO₃ and 2.5 mM Probenecid).

### FLIPR Assay —IC₅₀ Determination Protocol

For IC₅₀ determinations the fluorescence is read using FLIPR filter 1 (em 520-545 nM). A cellular baseline recording is taken for 30 seconds, followed by a 20 μl addition of 10, titrated half-log concentrations of the test compound, yielding cellular concentration ranging from 3 μM to 0.1 nM. Data is collected every 2 seconds for a further 5 minutes prior to the addition of a VR1 agonist solution: either 50 nM solution of capsaicin or MES (2-[N-morpholino]ethanesulfonic acid) buffer (pH 5.2), by the FLIPR pipettor. The FLIPR continues to collect data for a further 4 minutes. Compounds having antagonistic properties against the hVR1 will inhibit the increase in intracellular calcium in response to the capsaicin addition. This consequently leading to a reduction in fluorescence signal and providing a reduced fluorescence reading, compared with no compound, buffer controls. Data is exported by the FLIPR program as a sum of fluorescence calculated under the curve upon the addition of capsaicin. Maximum inhibition, Hill slope and IC₅₀ data for each compound are generated.

### LIST OF ABBREVIATIONS

VR1 vanilloid receptor 1
IBS irritable bowel syndrome
IBD inflammatory bowel disease
GERD gastro-esophageal reflux disease
HEPES 4-(2-Hydroxyethyl)piperazine-1-ethanesulfonic acid
EGTA Ethylene glycol-bis(2-aminoethylether)-N,N,N′,N′-tetraacetic acid
EMBLA Skatron, Plate Cell Washer, from Molecular Devices company

### HBSS Hank's Balanced Salt Solution

MES (2-[N-Morphholino]ethanesulfonic acid) Hydrate, Sigma cat# M-5287
NUT Nutrient mixture F-12, medium for culturing cells

### MEM Minimal Eagle Medium

### Results

Typical IC₅₀ values as measured in the assays described above are 1 μM or less. In one aspect of the invention the IC₅₀ is below 750 nM. In another aspect of the invention the IC₅₀ is below 150 nM. In a further aspect of the invention the IC₅₀ is below 10 nM.

TABLE 2
Specimen results from the hVR1 FLIPR.

| Compound | |
|---|---|
| No. | IC₅₀ nM |
| 3 | 119 |
| 11 | 716 |

1. A compound of formula I 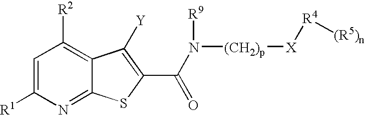
wherein:
R¹ and R² are independently selected from H, NO₂, NH₂, halo, N(C₁₋₃alkyl)₂, C₁₋₃alkyl, C₂₋₃alkenyl, C₂₋₃alkynyl, C₁₋₃haloalkyl, C₁₋₃haloalkylO, hydroxyC₁₋₃alkyl, C₁₋₃alkylOC₀₋₃alkyl, C₁₋₃alkylSC₀₋₃alkyl and C₁₋₃alkylNC₀₋₃alkyl;
Y is NH₂, NH(R³), N(R³)₂, OH, OR³ or NO₂;
R³ is C₁₋₃alkyl, C₂₋₃alkenyl, C₂₋₃alkynyl, C₁₋₃haloalkyl, C₁₋₃haloalkylO, hydroxyC₁₋₃alkyl, C₁₋₃alkylOC₀₋₃alkyl, C₁₋₃alkylSC₀₋₃alkyl or C₁₋₃alkylNC₀₋₃alkyl;
R⁹ is H, C₁₋₆alkyl, R⁶OC₀₋₆alkyl, or C₅₋₁₀arylC₀₋₆alkyl;
X is bond, CR⁶R⁷, NR⁶R⁷ or O;
p is 0, 1, 2, or 3;
R⁴ is bond, H, C₁₋₆alkyl, C₂₋₆alkenyl, C₂₋₆alkynyl, C₁₋₆haloalkyl, C₁₋₆haloalkylO, C₅₋₁₀arylC₀₋₆alkyl, C₅₋₁₀heteroarylC₀₋₆alkyl, C₃₋₁₅cycloalkylC₀₋₆alkyl, C₃₋₁₅heterocycloalkylC₀₋₆alkyl, R⁶OC₀₋₆alkyl, R⁶SC₀₋₆alkyl or R⁶NC₀₋₆alkyl, COOR⁶, R⁶COR⁷, R⁶CO₂, R⁶CONR⁷R⁸, R⁶NR⁷COC₀₋₆alkyl, R⁶SO₂R⁷ or R⁶SOR⁷R⁸;
R⁵ is H, OH, oxy, NO₂, NH₂, halo, N(C₁₋₃alkyl)₂, C₁₋₃alkyl, C₂₋₃alkenyl, C₂₋₃alkynyl, C₁₋₃haloalkyl, C₁₋₃haloalkylO, hydroxyC₁₋₃alkyl, R⁶OC₀₋₆alkyl, R⁶SC₀₋₆alkyl, R⁶NC₀₋₆alkyl, C₅₋₁₀arylOC₀₋₆alkyl, C₅₋₁₀-heteroarylOC₀₋₆alkyl, C₃₋₁₀cycloalkylOC₀₋₆alkyl, R⁶COO, R⁶COR⁷, R⁶CO₂, R⁶CONR⁷R⁸, R⁶NR⁷COC₀₋₆alkyl or R⁶SO₂R⁷ or R⁶SOR⁷R⁸;
R⁶, R⁷ and R⁸ are independently selected from H, C₁₋₆alkyl and C₅₋₁₀arylC₀₋₆alkyl;
or X and R⁶ form a 4, 5, 6 or 7 membered ring; and
n is 0, 1, 2, 3, 4, 5, 6 or 7;
or salts, solvates or solvated salts thereof.

2. A compound according to claim 1, wherein p is 1, 2, or 3, with the proviso that is not 3-amino-6-methyl-4-trifluoromethyl-thieno[2,3-b]pyridine-2-carboxylic acid benzylamide.

3. A compound selected from the group consisting of:
3-amino-6-methyl-N-(3-phenylpropyl)-4-(trifluoromethyl)thieno[2,3-b]pyridine-2-carboxamide,
3-amino-6-methyl-N-[2-(4-methylphenyl)ethyl]-4-(trifluoromethyl)thieno[2,3-b]pyridine-2-carboxamide,
3-amino-6-methyl-N-[2-(2-methylphenyl)ethyl]-4-(trifluoromethyl)thieno[2,3-b]pyridine-2-carboxamide,
3-amino-6-methyl-N-(2-phenylpropyl)-4-(trifluoromethyl)thieno[2,3-b]pyridine-2-carboxamide,
3-amino-N,6-dimethyl-N-(2-phenylethyl)-4-(trifluoromethyl)thieno[2,3-b]pyridine-2-carboxamide,
3-amino-N-[2-(2-methoxyphenyl)ethyl]-6-methyl-4-(trifluoromethyl)thieno[2,3-b]pyridine-2-carboxamide,
3-amino-N-(2,2-diphenylethyl)-6-methyl-4-(trifluoromethyl)thieno[2,3-b]pyridine-2-carboxamide,
3-amino-N-[2-(3-fluorophenyl)ethyl]-6-methyl-4-(trifluoromethyl)thieno[2,3-b]pyridine-2-carboxamide,
3-amino-N-[2-(3,4-dichlorophenyl)ethyl]-6-methyl-4-(trifluoromethyl)thieno[2,3-b]pyridine-2-carboxamide,
3-amino-6-methyl-4-(trifluoromethyl)-N-{2-[3-(trifluoromethyl)phenyl]ethyl}thieno[2,3-b]pyridine-2-carboxamide,
3-amino-6-methyl-N-{2-[3-(methyloxy)phenyl]ethyl}-4-(trifluoromethyl)thieno[2,3-b]pyridine-2-carboxamide,
3-amino-6-methyl-N-[2-(2-thienyl)ethyl]-4-(trifluoromethyl)thieno[2,3-b]pyridine-2-carboxamide,
3-amino-N-[2-(2,6-dichlorophenyl)ethyl]-6-methyl-4-(trifluoromethyl)thieno[2,3-b]pyridine-2-carboxamide,
3-amino-N-[2-(2-fluorophenyl)ethyl]-6-methyl-4-(trifluoromethyl)thieno[2,3-b]pyridine-2-carboxamide,
3-amino-6-methyl-N-[2-(phenyloxy)ethyl]-4-(trifluoromethyl)thieno[2,3-b]pyridine-2-carboxamide,
3-amino-N-(2,3-dihydro-1,4-benzodioxin-2-ylmethyl)-6-methyl-4-(trifluoromethyl)thieno[2,3-b]pyridine-2-carboxamide,
3-amino-N-{2-[4-(ethyloxy)phenyl]ethyl}-6-methyl-4-(trifluoromethyl)thieno[2,3-b]pyridine-2-carboxamide,
3-amino-6-methyl-N-(4-methylcyclohexyl)-4-(trifluoromethyl)thieno[2,3-b]pyridine-2-carboxamide,
3-amino-6-methyl-N-{2-[2-(phenyloxy)phenyl]ethyl}-4-(trifluoromethyl)thieno[2,3-b]pyridine-2-carboxamide,
3-amino-6-methyl-N-{[5-methyl-2-(trifluoromethyl)-3-furanyl]methyl}-4-(trifluoromethyl)thieno[2,3-b]pyridine-2-carboxamide,
1,1-dimethylethyl 4-({[3-amino-6-methyl-4-(trifluoromethyl)thieno[2,3-b]pyridin-2-yl]carbonyl}amino)-1-piperidinecarboxylate,
3-amino-N-{[3-fluoro-5-(trifluoromethyl)phenyl]methyl}-6-methyl-4-(trifluoromethyl)thieno[2,3-b]pyridine-2-carboxamide,
3-amino-6-methyl-4-(trifluoromethyl)-N-{[3-(trifluoromethyl)phenyl]methyl}thieno[2,3-b]pyridine-2-carboxamide,
3-amino-N-(3,3-dimethylbutyl)-6-methyl-4-(trifluoromethyl)thieno[2,3-b]pyridine-2-carboxamide,
3-amino-6-methyl-4-(trifluoromethyl)-N-({3-[(trifluoromethyl)oxy]phenyl}methyl)thieno[2,3-b]pyridine-2-carboxamide,
3-amino-N-{2-[4-(1,1-dimethylethyl)phenyl]ethyl}-6-methyl-4-(trifluoromethyl)thieno[2,3-b]pyridine-2-carboxamide,
3-amino-6-methyl-N-{3-[methyl(phenyl)amino]propyl}-4-(trifluoromethyl)thieno[2,3-b]pyridine-2-carboxamide,
3-amino-N-[(3,5-dimethylphenyl)methyl]-6-methyl-4-(trifluoromethyl)thieno[2,3-b]pyridine-2-carboxamide,
3-amino-N-(cyclohexylmethyl)-6-methyl-4-(trifluoromethyl)thieno[2,3-b]pyridine-2-carboxamide,
3-amino-N-butyl-6-methyl-4-(trifluoromethyl)thieno[2,3-b]pyridine-2-carboxamide,
3-amino-N-[2-(2,4-dichlorophenyl)ethyl]-6-methyl-4-(trifluoromethyl)thieno[2,3-b]pyridine-2-carboxamide,
3-amino-N-cyclohexyl-6-methyl-4-(trifluoromethyl)thieno[2,3-b]pyridine-2-carboxamide,
3-amino-N-[(5-fluoro-2-methylphenyl)methyl]-6-methyl-4-(trifluoromethyl)thieno[2,3-b]pyridine-2-carboxamide,
3-amino-N-[1-(4-fluorophenyl)ethyl]-6-methyl-4-(trifluoromethyl)thieno[2,3-b]pyridine-2-carboxamide,
3-amino-6-methyl-N-(2-methylpropyl)-4-(trifluoromethyl)thieno[2,3-b]pyridine-2-carboxamide,
3-amino-N-[(6-fluoro-4H-1,3-benzodioxin-8-yl)methyl]-6-methyl-4-(trifluoromethyl)thieno[2,3-b]pyridine-2-carboxamide,
3-amino-N,6-dimethyl-4-(trifluoromethyl)-N-{[3-(trifluoromethyl)phenyl]methyl}thieno[2,3-b]pyridine-2-carboxamide,
3-amino-N-(2,3-dihydro-1-benzofuran-5-ylmethyl)-6-methyl-4-(trifluoromethyl)thieno[2,3-b]pyridine-2-carboxamide,
3-amino-6-methyl-N-[2-(2-pyridinyl)ethyl]-4-(trifluoromethyl)thieno[2,3-b]pyridine-2-carboxamide,
3-amino-6-methyl-N-[2-(4-pyridinyl)ethyl]-4-(trifluoromethyl)thieno[2,3-b]pyridine-2-carboxamide,
3-amino-6-methyl-N-[(2S)-2-phenylpropyl]-4-(trifluoromethyl)thieno[2,3-b]pyridine-2-carboxamide,
3-amino-6-methyl-N-[(2R)-2-phenylpropyl]-4-(trifluoromethyl)thieno[2,3-b]pyridine-2-carboxamide,
3-amino-N-[(2R)-2-hydroxy-2-phenylethyl]-6-methyl-4-(trifluoromethyl)thieno[2,3-b]pyridine-2-carboxamide,
3-amino-N-[(2S)-2-hydroxy-2-phenylethyl]-6-methyl-4-(trifluoromethyl)thieno[2,3-b]pyridine-2-carboxamide,
3-amino-N-(2-hydroxy-2-phenylpropyl)-6-methyl-4-(trifluoromethyl)thieno[2,3-b]pyridine-2-carboxamide,
3-amino-N-[2-(2-furyl)ethyl]-6-methyl-4-(trifluoromethyl)thieno[2,3-b]pyridine-2-carboxamide,
3-amino-N-[2-(4-fluorophenyl)ethyl]-6-methyl-4-(trifluoromethyl)thieno[2,3-b]pyridine-2-carboxamide,
3-amino-N-(2-cyclohexylethyl)-6-methyl-4-(trifluoromethyl)thieno[2,3-b]pyridine-2-carboxamide,
3-amino-6-methyl-N-(trans-4-methylcyclohexyl)-4-(trifluoromethyl)thieno[2,3-b]pyridine-2-carboxamide,
6-methyl-3-(methylamino)-N-(2-phenylethyl)-4-(trifluoromethyl)thieno[2,3-b]pyridine-2-carboxamide,
3-(dimethylamino)-6-methyl-N-(2-phenylethyl)-4-(trifluoromethyl)thieno[2,3-b]pyridine-2-carboxamide,
3-amino-N-(2-phenylethyl)-4-(trifluoromethyl)thieno[2,3-b]pyridine-2-carboxamide,
3-amino-N-[2-(4-methylphenyl)ethyl]-4-(trifluoromethyl)thieno[2,3-b]pyridine-2-carboxamide, and
3-amino-N-[2-(3-fluorophenyl)ethyl]-4-(trifluoromethyl)thieno[2,3-b]pyridine-2-carboxamide,
or salts, solvates or solvated salts thereof.

4. A compound selected from the group consisting of
3-amino-6-methyl-N-(3-phenylpropyl)-4-(trifluoromethyl)thieno[2,3-b]pyridine-2-carboxamide,
3-amino-6-methyl-N-[2-(4-methylphenyl)ethyl]-4-(trifluoromethyl)thieno[2,3-b]pyridine-2-carboxamide,
3-amino-6-methyl-N-[2-(2-methylphenyl)ethyl]-4-(trifluoromethyl)thieno[2,3-b]pyridine-2-carboxamide,
3-amino-6-methyl-N-(2-phenylpropyl)-4-(trifluoromethyl)thieno[2,3-b]pyridine-2-carboxamide,
3-amino-N,6-dimethyl-N-(2-phenylethyl)-4-(trifluoromethyl)thieno[2,3-b]pyridine-2-carboxamide,
3-amino-N-[2-(2-methoxyphenyl)ethyl]-6-methyl-4-(trifluoromethyl)thieno[2,3-b]pyridine-2-carboxamide,
3-amino-N-(2,2-diphenylethyl)-6-methyl-4-(trifluoromethyl)thieno[2,3-b]pyridine-2-carboxamide,
3-amino-N-[2-(3-fluorophenyl)ethyl]-6-methyl-4-(trifluoromethyl)thieno[2,3-b]pyridine-2-carboxamide,
3-amino-N-[2-(3,4-dichlorophenyl)ethyl]-6-methyl-4-(trifluoromethyl)thieno[2,3-b]pyridine-2-carboxamide,
3-amino-6-methyl-4-(trifluoromethyl)-N-{2-[3-(trifluoromethyl)phenyl]ethyl}thieno[2,3-b]pyridine-2-carboxamide, and
or salts, solvates or solvated salts thereof.

5. A compound selected from the group consisting of:
3-amino-6-methyl-N-{2-[3-(methyloxy)phenyl]ethyl}-4-(trifluoromethyl)thieno[2,3-b]pyridine-2-carboxamide,
3-amino-6-methyl-N-[2-(2-thienyl)ethyl]-4-(trifluoromethyl)thieno[2,3-b]pyridine-2-carboxamide,
3-amino-N-[2-(2,6-dichlorophenyl)ethyl]-6-methyl-4-(trifluoromethyl)thieno[2,3-b]pyridine-2-carboxamide,
3-amino-N-[2-(2-fluorophenyl)ethyl]-6-methyl-4-(trifluoromethyl)thieno[2,3-b]pyridine-2-carboxamide,
3-amino-6-methyl-N-[2-(phenyloxy)ethyl]-4-(trifluoromethyl)thieno[2,3-b]pyridine-2-carboxamide,
3-amino-N-(2,3-dihydro-1,4-benzodioxin-2-ylmethyl)-6-methyl-4-(trifluoromethyl)thieno[2,3-b]pyridine-2-carboxamide,
3-amino-N-{2-[4-(ethyloxy)phenyl]ethyl}-6-methyl-4-(trifluoromethyl)thieno[2,3-b]pyridine-2-carboxamide,
3-amino-6-methyl-N-(4-methylcyclohexyl)-4-(trifluoromethyl)thieno[2,3-b]pyridine-2-carboxamide,
3-amino-6-methyl-N-{2-[2-(phenyloxy)phenyl]ethyl}-4-(trifluoromethyl)thieno[2,3-b]pyridine-2-carboxamide,
3-amino-6-methyl-N-{[5-methyl-2-(trifluoromethyl)-3-furanyl]methyl}-4-(trifluoromethyl)thieno[2,3-b]pyridine-2-carboxamide,
1,1-dimethylethyl 4-({[3-amino-6-methyl-4-(trifluoromethyl)thieno[2,3-b]pyridin-2-yl]carbonyl}amino)-1-piperidinecarboxylate,
3-amino-N-{[3-fluoro-5-(trifluoromethyl)phenyl]methyl}-6-methyl-4-(trifluoromethyl)thieno[2,3-b]pyridine-2-carboxamide,
3-amino-6-methyl-4-(trifluoromethyl)-N-{[3-(trifluoromethyl)phenyl]methyl}thieno[2,3-b]pyridine-2-carboxamide,
3-amino-N-(3,3-dimethylbutyl)-6-methyl-4-(trifluoromethyl)thieno[2,3-b]pyridine-2-carboxamide,
3-amino-6-methyl-4-(trifluoromethyl)-N-({3-[(trifluoromethyl)oxy]phenyl}methyl)thieno[2,3-b]pyridine-2-carboxamide,
3-amino-N-{2-[4-(1,1-dimethylethyl)phenyl]ethyl}-6-methyl-4-(trifluoromethyl)thieno[2,3-b]pyridine-2-carboxamide,
3-amino-6-methyl-N-{3-[methyl(phenyl)amino]propyl}-4-(trifluoromethyl)thieno[2,3-b]pyridine-2-carboxamide,
3-amino-N-[(3,5-dimethylphenyl)methyl]-6-methyl-4-(trifluoromethyl)thieno[2,3-b]pyridine-2-carboxamide,
3-amino-N-(cyclohexylmethyl)-6-methyl-4-(trifluoromethyl)thieno[2,3-b]pyridine-2-carboxamide,
3-amino-N-butyl-6-methyl-4-(trifluoromethyl)thieno[2,3-b]pyridine-2-carboxamide,
3-amino-N-[2-(2,4-dichlorophenyl)ethyl]-6-methyl-4-(trifluoromethyl)thieno[2,3-b]pyridine-2-carboxamide,
3-amino-N-cyclohexyl-6-methyl-4-(trifluoromethyl)thieno[2,3-b]pyridine-2-carboxamide,
3-amino-N-[(5-fluoro-2-methylphenyl)methyl]-6-methyl-4-(trifluoromethyl)thieno[2,3-b]pyridine-2-carboxamide,
3-amino-N-[1-(4-fluorophenyl)ethyl]-6-methyl-4-(trifluoromethyl)thieno[2,3-b]pyridine-2-carboxamide,
3-amino-6-methyl-N-(2-methylpropyl)-4-(trifluoromethyl)thieno[2,3-b]pyridine-2-carboxamide,
3-amino-N-[(6-fluoro-4H-1,3-benzodioxin-8-yl)methyl]-6-methyl-4-(trifluoromethyl)thieno[2,3-b]pyridine-2-carboxamide,
3-amino-N,6-dimethyl-4-(trifluoromethyl)-N-{[3-(trifluoromethyl)phenyl]methyl}thieno[2,3-b]pyridine-2-carboxamide,
3-amino-N-(2,3-dihydro-1-benzofuran-5-ylmethyl)-6-methyl-4-(trifluoromethyl)thieno[2,3-b]pyridine-2-carboxamide,
3-amino-6-methyl-N-[2-(2-pyridinyl)ethyl]-4-(trifluoromethyl)thieno[2,3-b]pyridine-2-carboxamide, and
3-amino-6-methyl-N-[2-(4-pyridinyl)ethyl]-4-(trifluoromethyl)thieno[2,3-b]pyridine-2-carboxamide,
or salts, solvates or solvated salts thereof.

6. A compound selected from the group consisting of:
3-amino-6-methyl-N-[(2S)-2-phenylpropyl]-4-(trifluoromethyl)thieno[2,3-b]pyridine-2-carboxamide,
3-amino-6-methyl-N-[(2R)-2-phenylpropyl]-4-(trifluoromethyl)thieno[2,3-b]pyridine-2-carboxamide,
3-amino-N-[(2R)-2-hydroxy-2-phenylethyl]-6-methyl-4-(trifluoromethyl)thieno[2,3-b]pyridine-2-carboxamide,
3-amino-N-[(2S)-2-hydroxy-2-phenylethyl]-6-methyl-4-(trifluoromethyl)thieno[2,3-b]pyridine-2-carboxamide,
3-amino-N-(2-hydroxy-2-phenylpropyl)-6-methyl-4-(trifluoromethyl)thieno[2,3-b]pyridine-2-carboxamide,
3-amino-N-[2-(2-furyl)ethyl]-6-methyl-4-(trifluoromethyl)thieno[2,3-b]pyridine-2-carboxamide,
3-amino-N-[2-(4-fluorophenyl)ethyl]-6-methyl-4-(trifluoromethyl)thieno[2,3-b]pyridine-2-carboxamide,
3-amino-N-(2-cyclohexylethyl)-6-methyl-4-(t-difluoromethyl)thieno[2,3-b]pyridine-2-carboxamide,
3-amino-6-methyl-N-(trans-4-methylcyclohexyl)-4-(trifluoromethyl)thieno[2,3-b]pyridine-2-carboxamide,
6-methyl-3-(methylamino)-N-(2-phenylethyl)-4-(trifluoromethyl)thieno[2,3-b]pyridine-2-carboxamide,
3-(dimethylamino)-6-methyl-N-(2-phenylethyl)-4-(trifluoromethyl)thieno[2,3-b]pyridine-2-carboxamide,
3-amino-N-(2-phenylethyl)-4-(trifluoromethyl)thieno[2,3-b]pyridine-2-carboxamide,
3-amino-N-[2-(4-methylphenyl)ethyl]-4-(trifluoromethyl)thieno[2,3-b]pyridine-2-carboxamide, and
3-amino-N-[2-(3-fluorophenyl)ethyl]-4-(trifluoromethyl)thieno[2,3-b]pyridine-2-carboxamide,
or salts, solvates or solvated salts thereof.

7. A compound according to claim 1, for use in therapy.

8. A compound according to claim 1, in treatment of VR1 mediated disorders.

9. A compound according to claim 8 for treatment of acute and chronic pain, acute and chronic neuropathic pain and acute and chronic inflammatory pain.

10. A compound according to claim 8 for treatment of respiratory diseases.

11. A method of treatment of VR1 mediated disorders and for treatment of acute and chronic pain, acute and chronic neuropathic pain and acute and chronic inflammatory pain, and respiratory diseases, comprising
administering to a mammal, including man in need of such treatment, a therapeutically effective amount of the compound of formula I, according to claim 1.

12. A pharmaceutical formulation comprising as active ingredient a therapeutically effective amount of the compound of formula I, according to claim 1, in association with one or more pharmaceutically acceptable diluents, excipients and/or inert carriers.

13. The pharmaceutical formulation according to claim 12, for use in the treatment of VR1 mediated disorders and for treatment of acute and chronic pain, acute and chronic neuropathic pain and acute and chronic inflammatory pain, and respiratory diseases.

14. The compound 3-amino-6-methyl-4-(trifluoromethyl)thieno[2,3-b]pyridine-2-carboxylic acid used as an intermediate in the preparation of a compound according to any claim 1.

15. A compound selected from:
4-(trifluoromethyl)nicotinonitrile 1-oxide,
2-chloro-4-(trifluoromethyl)nicotinonitrile, and
3-amino-4-(trifluoromethyl)thieno[2,3-b]pyridine-2-carboxylic acid.

16. A compound according to claim 15 used as an intermediate in the preparation of a compound of formula I.
